SEQUENCE LISTING (256 sequences)

SEQ ID NO: 1 - protein, 8 aa, Artificial Sequence
  protease cleavage site
AHPRIISA

SEQ ID NO: 2 - protein, 8 aa, Artificial Sequence
  protease cleavage site
ARTRARRP

SEQ ID NO: 3 - protein, 8 aa, Artificial Sequence
  protease cleavage site
ASARTTGS

SEQ ID NO: 4 - protein, 8 aa, Artificial Sequence
  protease cleavage site
ATPRGAAP

SEQ ID NO: 5 - protein, 8 aa, Artificial Sequence
  protease cleavage site
AVVRTPPK

SEQ ID NO: 6 - protein, 8 aa, Artificial Sequence
  protease cleavage site
ERTRSFQL

SEQ ID NO: 7 - protein, 8 aa, Artificial Sequence
  protease cleavage site
FGLRFYAY

SEQ ID NO: 8 - protein, 8 aa, Artificial Sequence
  protease cleavage site
FNPRTFGS

SEQ ID NO: 9 - protein, 8 aa, Artificial Sequence
  protease cleavage site
FRPKHTRI

SEQ ID NO: 10 - protein, 8 aa, Artificial Sequence
  protease cleavage site
FSARGHRP

SEQ ID NO: 11 - protein, 8 aa, Artificial Sequence
  protease cleavage site
GDIRGPRI

SEQ ID NO: 12 - protein, 8 aa, Artificial Sequence
  protease cleavage site
GGVRGPRV

SEQ ID NO: 13 - protein, 8 aa, Artificial Sequence
  protease cleavage site
GSFRAGLF

SEQ ID NO: 14 - protein, 8 aa, Artificial Sequence
  protease cleavage site
HKGRSALV

SEQ ID NO: 15 - protein, 8 aa, Artificial Sequence
  protease cleavage site
IAGRSLNP

SEQ ID NO: 16 - protein, 8 aa, Artificial Sequence
  protease cleavage site
IDGRIVEG

SEQ ID NO: 17 - protein, 8 aa, Artificial Sequence
  protease cleavage site
IEPRSFSQ

SEQ ID NO: 18 - protein, 8 aa, Artificial Sequence
  protease cleavage site
IKPRIVGG

SEQ ID NO: 19 - protein, 8 aa, Artificial Sequence
  protease cleavage site
IQIRSVAK

SEQ ID NO: 20 - protein, 8 aa, Artificial Sequence
  protease cleavage site
IQIRSVAK

SEQ ID NO: 21 - protein, 8 aa, Artificial Sequence
  protease cleavage site
KNVKSKIG

SEQ ID NO: 22 - protein, 8 aa, Artificial Sequence
  protease cleavage site
KPKDSSVD

SEQ ID NO: 23 - protein, 8 aa, Artificial Sequence
  protease cleavage site
LDPRSFLL

SEQ ID NO: 24 - protein, 8 aa, Artificial Sequence
  protease cleavage site
LGIRSFRN

SEQ ID NO: 25 - protein, 8 aa, Artificial Sequence
  protease cleavage site
LPIKTFRG

SEQ ID NO: 26 - protein, 8 aa, Artificial Sequence
  protease cleavage site
LRPRFKII

SEQ ID NO: 27 - protein, 8 aa, Artificial Sequence
  protease cleavage site
LRPRIIGG

SEQ ID NO: 28 - protein, 8 aa, Artificial Sequence
  protease cleavage site
LSPRGVHI

SEQ ID NO: 29 - protein, 8 aa, Artificial Sequence
  protease cleavage site
LSPRTFHP

SEQ ID NO: 30 - protein, 8 aa, Artificial Sequence
  protease cleavage site
MTPRSEGS

SEQ ID NO: 31 - protein, 8 aa, Artificial Sequence
  protease cleavage site
MTPRSGGS

SEQ ID NO: 32 - protein, 8 aa, Artificial Sequence
  protease cleavage site
MTPRSRGS

SEQ ID NO: 33 - protein, 8 aa, Artificial Sequence
  protease cleavage site
MVPRAVYL

SEQ ID NO: 34 - protein, 8 aa, Artificial Sequence
  protease cleavage site
NVPRILSP

SEQ ID NO: 35 - protein, 8 aa, Artificial Sequence
  protease cleavage site
PAPRGYPG

SEQ ID NO: 36 - protein, 8 aa, Artificial Sequence
  protease cleavage site
PDLRSCVN

SEQ ID NO: 37 - protein, 8 aa, Artificial Sequence
  protease cleavage site
PGPRGPPP

SEQ ID NO: 38 - protein, 8 aa, Artificial Sequence
  protease cleavage site
PGSRSRTP

SEQ ID NO: 39 - protein, 8 aa, Artificial Sequence
  protease cleavage site
PQGRIVGG

SEQ ID NO: 40 - protein, 8 aa, Artificial Sequence
  protease cleavage site
PQGRTTAH

SEQ ID NO: 41 - protein, 8 aa, Artificial Sequence
  protease cleavage site
PRSFLLRN

SEQ ID NO: 42 - protein, 8 aa, Artificial Sequence
  protease cleavage site
QSPRSFQK

SEQ ID NO: 43 - protein, 8 aa, Artificial Sequence
  protease cleavage site
QSPRSFQK

SEQ ID NO: 44 - protein, 8 aa, Artificial Sequence
  protease cleavage site
QYLRVPLV

SEQ ID NO: 45 - protein, 8 aa, Artificial Sequence
  protease cleavage site
SEFRCLTP

SEQ ID NO: 46 - protein, 8 aa, Artificial Sequence
  protease cleavage site
SIGRASLH

SEQ ID NO: 47 - protein, 8 aa, Artificial Sequence
  protease cleavage site
SKGRSLIG

SEQ ID NO: 48 - protein, 8 aa, Artificial Sequence
  protease cleavage site
SRLRAYLL

SEQ ID NO: 49 - protein, 8 aa, Artificial Sequence
  protease cleavage site
TCLRSTKF

SEQ ID NO: 50 - protein, 8 aa, Artificial Sequence
  protease cleavage site
VCLRSFQT

SEQ ID NO: 51 - protein, 8 aa, Artificial Sequence
  protease cleavage site
VDPRLIDG

SEQ ID NO: 52 - protein, 8 aa, Artificial Sequence
  protease cleavage site
VEVKSEKL

SEQ ID NO: 53 - protein, 8 aa, Artificial Sequence
  protease cleavage site
VIPKRISP

SEQ ID NO: 54 - protein, 8 aa, Artificial Sequence
  protease cleavage site
VIPRSGGS

SEQ ID NO: 55 - protein, 8 aa, Artificial Sequence
  protease cleavage site
VNPRGIVT

SEQ ID NO: 56 - protein, 8 aa, Artificial Sequence
  protease cleavage site
VQPRAQKI

SEQ ID NO: 57 - protein, 8 aa, Artificial Sequence
  protease cleavage site
VSPRASAS

SEQ ID NO: 58 - protein, 8 aa, Artificial Sequence
  protease cleavage site
VVPRGVNL

SEQ ID NO: 59 - protein, 8 aa, Artificial Sequence
  protease cleavage site
WYLRSNNG

SEQ ID NO: 60 - protein, 8 aa, Artificial Sequence
  protease cleavage site
WYLRSNTG

SEQ ID NO: 61 - protein, 8 aa, Artificial Sequence
  protease cleavage site
AFWKTDAS

SEQ ID NO: 62 - protein, 8 aa, Artificial Sequence
  protease cleavage site
IEGRTATS

SEQ ID NO: 63 - protein, 8 aa, Artificial Sequence
  protease cleavage site
TAARQSTN

SEQ ID NO: 64 - protein, 8 aa, Artificial Sequence
  protease cleavage site
IDGRIVEG

SEQ ID NO: 65 - protein, 8 aa, Artificial Sequence
  protease cleavage site
FNPRTFGS

SEQ ID NO: 66 - protein, 8 aa, Artificial Sequence
  protease cleavage site
RVPKSFPF

SEQ ID NO: 67 - protein, 8 aa, Artificial Sequence
  protease cleavage site
PQLRMKNN

SEQ ID NO: 68 - protein, 8 aa, Artificial Sequence
  protease cleavage site
SSWRLTSS

SEQ ID NO: 69 - protein, 8 aa, Artificial Sequence
  protease cleavage site
LGIRSFRN

SEQ ID NO: 70 - protein, 8 aa, Artificial Sequence
  protease cleavage site
AMSRMSLS

SEQ ID NO: 71 - protein, 8 aa, Artificial Sequence
  protease cleavage site
ANNRDNTY

SEQ ID NO: 72 - protein, 8 aa, Artificial Sequence
  protease cleavage site
ANVRRKRY

SEQ ID NO: 73 - protein, 8 aa, Artificial Sequence
  protease cleavage site
ARGRAFPQ

SEQ ID NO: 74 - protein, 8 aa, Artificial Sequence
  protease cleavage site
ATLKSRKM

SEQ ID NO: 75 - protein, 8 aa, Artificial Sequence
  protease cleavage site
ATQKKVER

SEQ ID NO: 76 - protein, 8 aa, Artificial Sequence
  protease cleavage site
ATWKTRWY

SEQ ID NO: 77 - protein, 8 aa, Artificial Sequence
  protease cleavage site
DVGEYNVF

SEQ ID NO: 78 - protein, 8 aa, Artificial Sequence
  protease cleavage site
EARGSVIL

SEQ ID NO: 79 - protein, 8 aa, Artificial Sequence
  protease cleavage site
EAYRRFYG

SEQ ID NO: 80 - protein, 8 aa, Artificial Sequence
  protease cleavage site
EDNRDSSM

SEQ ID NO: 81 - protein, 8 aa, Artificial Sequence
  protease cleavage site
ETLKVIDE

SEQ ID NO: 82 - protein, 8 aa, Artificial Sequence
  protease cleavage site
FRARAYGF

SEQ ID NO: 83 - protein, 8 aa, Artificial Sequence
  protease cleavage site
GEARGSVI

SEQ ID NO: 84 - protein, 8 aa, Artificial Sequence
  protease cleavage site
GGYRARPA

SEQ ID NO: 85 - protein, 8 aa, Artificial Sequence
  protease cleavage site
GILKENAA

SEQ ID NO: 86 - protein, 8 aa, Artificial Sequence
  protease cleavage site
GPKRGTEP

SEQ ID NO: 87 - protein, 8 aa, Artificial Sequence
  protease cleavage site
IIRRSIQI

SEQ ID NO: 88 - protein, 8 aa, Artificial Sequence
  protease cleavage site
ITFRMNVA

SEQ ID NO: 89 - protein, 8 aa, Artificial Sequence
  protease cleavage site
KHSKRHIH

SEQ ID NO: 90 - protein, 8 aa, Artificial Sequence
  protease cleavage site
KKDRARQE

SEQ ID NO: 91 - protein, 8 aa, Artificial Sequence
  protease cleavage site
KKPRCGVP

SEQ ID NO: 92 - protein, 8 aa, Artificial Sequence
  protease cleavage site
KQVKDNEN

SEQ ID NO: 93 - protein, 8 aa, Artificial Sequence
  protease cleavage site
LDPRSFLL

SEQ ID NO: 94 - protein, 8 aa, Artificial Sequence
  protease cleavage site
LPPKSQPP

SEQ ID NO: 95 - protein, 8 aa, Artificial Sequence
  protease cleavage site
LSFRARAY

SEQ ID NO: 96 - protein, 8 aa, Artificial Sequence
  protease cleavage site
MSMRVRRH

SEQ ID NO: 97 - protein, 8 aa, Artificial Sequence
  protease cleavage site
NSGRAVTY

SEQ ID NO: 98 - protein, 8 aa, Artificial Sequence
  protease cleavage site
PAPRGYPG

SEQ ID NO: 99 - protein, 8 aa, Artificial Sequence
  protease cleavage site
PEAKASCY

SEQ ID NO: 100 - protein, 8 aa, Artificial Sequence
  protease cleavage site
PESKATNA

SEQ ID NO: 101 - protein, 8 aa, Artificial Sequence
  protease cleavage site
PGPKRGTE

SEQ ID NO: 102 - protein, 8 aa, Artificial Sequence
  protease cleavage site
PKAKSHAP

SEQ ID NO: 103 - protein, 8 aa, Artificial Sequence
  protease cleavage site
PLDKKREE

SEQ ID NO: 104 - protein, 8 aa, Artificial Sequence
  protease cleavage site
PLQKQLPA

SEQ ID NO: 105 - protein, 8 aa, Artificial Sequence
  protease cleavage site
PQFRIKGG

SEQ ID NO: 106 - protein, 8 aa, Artificial Sequence
  protease cleavage site
PQLRLPHT

SEQ ID NO: 107 - protein, 8 aa, Artificial Sequence
  protease cleavage site
PQLRRGWR

SEQ ID NO: 108 - protein, 8 aa, Artificial Sequence
  protease cleavage site
PQSRSVPP

SEQ ID NO: 109 - protein, 8 aa, Artificial Sequence
  protease cleavage site
PRFKIIGG

SEQ ID NO: 110 - protein, 8 aa, Artificial Sequence
  protease cleavage site
PYLKVFNP

SEQ ID NO: 111 - protein, 8 aa, Artificial Sequence
  protease cleavage site
QKSRNGLR

SEQ ID NO: 112 - protein, 8 aa, Artificial Sequence
  protease cleavage site
QLIKAIQL

SEQ ID NO: 113 - protein, 8 aa, Artificial Sequence
  protease cleavage site
QRYKVDYE

SEQ ID NO: 114 - protein, 8 aa, Artificial Sequence
  protease cleavage site
RAQRSAGA

SEQ ID NO: 115 - protein, 8 aa, Artificial Sequence
  protease cleavage site
RGPRVVER

SEQ ID NO: 116 - protein, 8 aa, Artificial Sequence
  protease cleavage site
RPAKAAAT

SEQ ID NO: 117 - protein, 8 aa, Artificial Sequence
  protease cleavage site
RRKRYAIQ

SEQ ID NO: 118 - protein, 8 aa, Artificial Sequence
  protease cleavage site
RSSKGRSL

SEQ ID NO: 119 - protein, 8 aa, Artificial Sequence
  protease cleavage site
RSTRFAAT

SEQ ID NO: 120 - protein, 8 aa, Artificial Sequence
  protease cleavage site
SCDKTHTC

SEQ ID NO: 121 - protein, 8 aa, Artificial Sequence
  protease cleavage site
SINKSSPL

SEQ ID NO: 122 - protein, 8 aa, Artificial Sequence
  protease cleavage site
SQPPEKTE

SEQ ID NO: 123 - protein, 8 aa, Artificial Sequence
  protease cleavage site
SQRKHSKR

SEQ ID NO: 124 - protein, 8 aa, Artificial Sequence
  protease cleavage site
SSMKLSFR

SEQ ID NO: 125 - protein, 8 aa, Artificial Sequence
  protease cleavage site
TEPKVKLP

SEQ ID NO: 126 - protein, 8 aa, Artificial Sequence
  protease cleavage site
TEYRLVSI

SEQ ID NO: 127 - protein, 8 aa, Artificial Sequence
  protease cleavage site
THEKGRQS

SEQ ID NO: 128 - protein, 8 aa, Artificial Sequence
  protease cleavage site
TYSKASTP

SEQ ID NO: 129 - protein, 8 aa, Artificial Sequence
  protease cleavage site
TYSRSRYL

SEQ ID NO: 130 - protein, 8 aa, Artificial Sequence
  protease cleavage site
VSNKVSMS

SEQ ID NO: 131 - protein, 228 aa, Artificial Sequence
  Fab-1 light chain
SYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGGGGSLVPRGSGGGGSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 132 - protein, 16 aa, Artificial Sequence
  protease cleavage site and linker
GGGGSLVPRGSGGGGS

SEQ ID NO: 133 - protein, 245 aa, Artificial Sequence
  Fab-1 heavy chain
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSGGGGSLVPRGSGGGGSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVDYKDDDDKHHHHHH

SEQ ID NO: 134 - DNA, 1590 bp, Artificial Sequence
  sequence encoding Fab-1
ggtctcacatgaaaaaaaccgctatcgctatcgccgtcgcactggctggcttcgcaaccgtggcacaggcatcctatgaactgacccaaccgccgagtgtctccgtgtcaccgggtcagacggcacgtattacctgcagcggtgataacctgccgaaatattacgcgcattggtatcagcaaaaaccgggccaagccccggtggttgtcatcttttatgacgttaatcgtccgtccggtatcccggaacgcttctcgggcagcaactctggtaatacggcaaccctgacgatcagcggcacccaggcaatggatgaagctgactattactgtcaagcatggtggagctctacgccggtgtttggcggtggcacgaaactgaccgtgctgggtggcggtggctctctggttccgcgtggctccggtggcggtggctcaggccagccgaaagcagcaccgagtgttaccctgtttccgccgagttccgaagaactgcaagcaaacaaagctaccctggtgtgcctgattagcgatttctatccgggcgcagttacggtcgcgtggaaagccgactcatcgccggtgaaagctggtgttgaaaccacgaccccgtcaaaacagtcgaacaataaatatgcagctagctcttacctgtctctgaccccggaacagtggaaaagtcatcgcagttactcctgtcaagttacgcacgaaggctccacggtcgaaaaaaccgtggcaccgacggaatgctcatgataagcatgcgtaggagaaaataaaatgaaacagtctaccatcgcactggcactgctgccgctgctgtttacgccggtgaccaaagcagaagttcagctggtcgaaagtggtggcggtctggttcaaccgggcggttcactgcgtctgtcgtgcgcagcaagcggttttaccttcagttcctatggtatggattgggtccgtcaggcaccgggtaaaggtctggaatgggtgtcatcgattcgtggcagccgcggttctacctattacgccgattcagttaaaggccgttttaccatctctcgcgacaacagtaaaaatacgctgtatctgcagatgaacagcctgcgcgcggaagataccgccgtgtattactgtgcccgtctgtatcgctactggttcgactactggggccagggtacgctggtgaccgttagctctggcggtggcggttcgctggtcccgcgtggcagcggcggtggcggttcagcgagcaccaaaggtccgagcgtgtttccgctggcaccgtgcagccgctctaccagtgaatccacggcagctctgggttgtctggtgaaagattattttccggaaccggtcaccgtgagttggaactccggcgcactgacctcgggtgttcatacgttcccggctgtcctgcagagttccggcctgtatagcctgtcatcggtggttaccgttccgagctctaatttcggcacccaaacgtacacctgcaacgtcgatcacaaaccgtctaataccaaagttgacaaaacggttgattacaaagacgacgacgacaaacaccaccaccaccaccattgataagctt

SEQ ID NO: 135 - protein, 218 aa, Artificial Sequence
  Fab-2 light chain
SYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLLVPRGSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 136 - protein, 235 aa, Artificial Sequence
  Fab-2 Heavy Chain
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSLVPRGSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVDYKDDDDKHHHHHH

SEQ ID NO: 137 - protein, 6 aa, Artificial Sequence
  protease cleavage site
LVPRGS

SEQ ID NO: 138 - DNA, 1530 bp, Artificial Sequence
  Fab-2 coding sequence
ggtctcacatgaaaaaaacggctatcgcaatcgctgtggcactggcaggcttcgcaacggtcgcgcaggcatcctatgaactgacgcaaccgccgagtgtctccgtgtcaccgggtcagacggcacgtattacctgctctggtgataacctgccgaaatattacgcgcattggtatcagcaaaaaccgggccaagccccggtggttgtcatcttttatgacgttaatcgtccgtcgggtatcccggaacgcttctcgggcagcaactctggtaatacggccaccctgacgatctcaggcacccaggcaatggatgaagctgactattactgccaagcatggtggagctctacgccggtgtttggcggtggcacgaaactgaccgttctgctggtcccgcgtggctccggtcagccgaaagcagccccgtcagttaccctgtttccgccgagttccgaagaactgcaagcaaacaaagctaccctggtgtgtctgattagcgatttctatccgggcgcagttacggtcgcgtggaaagccgactcatcgccggtcaaagctggtgtggaaaccaccaccccgtcaaaacagtcgaacaataaatatgcagctagctcttacctgtcgctgaccccggaacagtggaaaagccatcgcagttactcctgccaagttacgcacgaaggctctacggttgaaaaaaccgtcgcaccgacggaatgcagttgataagcatgcgtaggagaaaataaaatgaaacagagcaccatcgcactggcactgctgccgctgctgtttacgccggtcaccaaagcagaagtgcagctggttgaatctggtggcggtctggtgcaaccgggcggttcactgcgtctgtcgtgtgcggccagcggctttaccttcagttcctatggtatggattgggtccgtcaggcaccgggtaaaggtctggaatgggtgtcatcgattcgtggcagccgcggttctacctattacgccgattccgttaaaggccgtttcaccatctctcgcgacaacagtaaaaatacgctgtatctgcagatgaacagtctgcgcgcggaagataccgccgtgtattactgcgcccgtctgtatcgctactggtttgactactggggccagggtacgctggtgaccgttagctctctggttccgcgtggctcagcgagcaccaaaggtccgagtgtcttcccgctggcaccgtgcagccgctctaccagtgaatccacggcagctctgggttgtctggtgaaagattattttccggaaccggtcaccgtgagttggaactccggcgcactgacctccggtgtgcatacgttcccggctgttctgcagagttccggcctgtattcactgtcatcggtggttaccgtgccgagctctaattttggcacccaaacgtacacctgtaacgttgatcacaaaccgagcaataccaaagttgacaaaaccgttgactacaaagacgacgacgacaaacaccaccaccaccaccactgataagctt

SEQ ID NO: 139 - protein, 228 aa, Artificial Sequence; identical to SEQ ID NO: 131
  IgG2-linker 1, Light chain amino acid sequence

SEQ ID NO: 140 - protein, 458 aa, Artificial Sequence
  IgG2-linker 1, heavy chain amino acid sequence
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSGGGGSLVPRGSGGGGSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 141 - DNA, 684 bp, Artificial Sequence
  IgG2-linker 1, Light chain DNA sequence
tcctacgagctgacccagcccccttccgtgtccgtgtctcctggccagaccgcccggatcacctgttccggcgacaacctgcccaagtactacgcccactggtatcagcagaagcccggccaggcccccgtggtggtcatcttctacgacgtgaaccggccctccggcatccccgagagattctccggctccaactccggcaacaccgccaccctgaccatctccggcacccaggccatggacgaggccgactactactgccaggcttggtggtcctccacccccgtgtttggcggcggaacaaagttaaccgtgctgggcggtggaggatcactggttccgcgtggctctggcggtggaggatcaggccagcccaaggccgctccttccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaggtcacacacgagggctccaccgtggaaaagacagtggcccccaccgagtgctct

SEQ ID NO: 142 - DNA, 1374 bp, Artificial Sequence
  IgG2-linker 1, Heavy chain DNA sequence
gaggtgcagctggtggaatccggcggaggcctggtccagcctggcggatctctgagactgtcctgcgccgcctccggcttcaccttctccagctacggcatggactgggtccgacaggcccctggcaagggcctggaatgggtgtcctccatccggggctctcggggctccacctactacgccgactccgtgaagggccggttcaccatctcccgggacaactccaagaacaccctgtacctgcagatgaactccctgcgggccgaggacaccgccgtgtactactgcgccagactgtaccggtattggttcgactactggggccagggcaccctggtcaccgtcagctcaggcggtggaggatcactggttccgcgtggctctggcggtggaggatcagcttccaccaagggcccctccgtgttccctctggccccttgctcccggtccacctctgagtctaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgacagtgccctcctccaacttcggcacccagacctacacctgtaacgtggaccacaagccctccaacaccaaggtggacaagaccgtggaacggaagtgctgcgtggaatgcccaccctgtcctgctccacctgtggctggccccagcgtgttcctgttccccccaaagcccaaggacaccctgatgatctcccggacccccgaagtgacctgcgtggtggtggacgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcatcaggactggctgaacggcaaagagtacaagtgcaaggtctccaacaagggcctgcctgcccccatcgaaaagaccatcagcaagaccaagggccagccccgcgagccccaggtgtacacactgccccccagccgggaagagatgaccaagaaccaggtgtccctgacctgtctggtgaaaggcttctacccctccgacattgccgtggaatgggagtccaacggacagcctgagaacaactacaagaccaccccccccatgctggactccgacggctcattcttcctgtactccaagctgacagtggacaagtcccggtggcagcagggcaacgtgttctcctgctccgtgatgcacgaggccctgcacaaccactacacccagaagtccctgtccctgagccccggc

SEQ ID NO: 143 - protein, 218 aa, Artificial Sequence; identical to SEQ ID NO: 135
  IgG2-linker 2, Light chain amino acid sequence

SEQ ID NO: 144 - protein, 448 aa, Artificial Sequence
  IgG2-linker 2, heavy chain amino acid sequence
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSLVPRGSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 145 - DNA, 654 bp, Artificial Sequence
  IgG2-linker 2, Light chain DNA sequence
tcctacgagctgacccagcccccttccgtgtccgtgtctcctggccagaccgcccggatcacctgttccggcgacaacctgcccaagtactacgcccactggtatcagcagaagcccggccaggcccccgtggtggtcatcttctacgacgtgaaccggccctccggcatccccgagagattctccggctccaactccggcaacaccgccaccctgaccatctccggcacccaggccatggacgaggccgactactactgccaggcttggtggtcctccacccccgtgtttggcggcggaacaaagttaaccgtgctgctggttccgcgtggctctggccagcccaaggccgctccttccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaggtcacacacgagggctccaccgtggaaaagacagtggcccccaccgagtgctct

SEQ ID NO: 146 - DNA, 1344 bp, Artificial Sequence
  IgG2-linker 2, Heavy chain DNA sequence
gaggtgcagctggtggaatccggcggaggcctggtccagcctggcggatctctgagactgtcctgcgccgcctccggcttcaccttctccagctacggcatggactgggtccgacaggcccctggcaagggcctggaatgggtgtcctccatccggggctctcggggctccacctactacgccgactccgtgaagggccggttcaccatctcccgggacaactccaagaacaccctgtacctgcagatgaactccctgcgggccgaggacaccgccgtgtactactgcgccagactgtaccggtattggttcgactactggggccagggcaccctggtcaccgtcagctcactggttccgcgtggctctgcttccaccaagggcccctccgtgttccctctggccccttgctcccggtccacctctgagtctaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgacagtgccctcctccaacttcggcacccagacctacacctgtaacgtggaccacaagccctccaacaccaaggtggacaagaccgtggaacggaagtgctgcgtggaatgcccaccctgtcctgctccacctgtggctggccccagcgtgttcctgttccccccaaagcccaaggacaccctgatgatctcccggacccccgaagtgacctgcgtggtggtggacgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcatcaggactggctgaacggcaaagagtacaagtgcaaggtctccaacaagggcctgcctgcccccatcgaaaagaccatcagcaagaccaagggccagccccgcgagccccaggtgtacacactgccccccagccgggaagagatgaccaagaaccaggtgtccctgacctgtctggtgaaaggcttctacccctccgacattgccgtggaatgggagtccaacggacagcctgagaacaactacaagaccaccccccccatgctggactccgacggctcattcttcctgtactccaagctgacagtggacaagtcccggtggcagcagggcaacgtgttctcctgctccgtgatgcacgaggccctgcacaaccactacacccagaagtccctgtccctgagccccggc

SEQ ID NO: 147 - protein, 6 aa, Artificial Sequence
  Thrombin cleavage site
  VARIANT (1)..(2): Xaa = hydrophobic amino acid
  VARIANT (5)..(6): Xaa = nonacidic amino acid
XXPRXX

SEQ ID NO: 148 - protein, 4 aa, Artificial Sequence
  Factor Xa cleavage site
  VARIANT (2)..(2): Xaa = Glu or Asp
IXGR

SEQ ID NO: 149 - protein, 5 aa, Artificial Sequence
  amino acid linker
GGGGS

SEQ ID NO: 150 - protein, 58 aa, Artificial Sequence
  amino acids 95-152 of SEQ ID NO:133
YCARLYRYWFDYWGQGTLVTVSSASGSGGGLVPRGSGGGGSTKGPSVFPLAPCSRSTS

SEQ ID NO: 151 - protein, 16 aa, Artificial Sequence
  amino acid linker
AKTTPKLEEGEFSEAR

SEQ ID NO: 152 - protein, 17 aa, Artificial Sequence
  amino acid linker
AKTTPKLEEGEFSEARV

SEQ ID NO: 153 - protein, 9 aa, Artificial Sequence
  amino acid linker
AKTTPKLGG

SEQ ID NO: 154 - protein, 10 aa, Artificial Sequence
  amino acid linker
SAKTTPKLGG

SEQ ID NO: 155 - protein, 6 aa, Artificial Sequence
  amino acid linker
SAKTTP

SEQ ID NO: 156 - protein, 6 aa, Artificial Sequence
  amino acid linker
RADAAP

SEQ ID NO: 157 - protein, 9 aa, Artificial Sequence
  amino acid linker
RADAAPTVS

SEQ ID NO: 158 - protein, 12 aa, Artificial Sequence
  amino acid linker
RADAAAAGGPGS

SEQ ID NO: 159 - protein, 27 aa, Artificial Sequence
  amino acid linker
RADAAAAGGGGSGGGGSGGGGSGGGGS

SEQ ID NO: 160 - protein, 18 aa, Artificial Sequence
  amino acid linker
SAKTTPKLEEGEFSEARV

SEQ ID NO: 161 - protein, 5 aa, Artificial Sequence
  amino acid linker
ADAAP

SEQ ID NO: 162 - protein, 12 aa, Artificial Sequence
  amino acid linker
ADAAPTVSIFPP

SEQ ID NO: 163 - protein, 5 aa, Artificial Sequence
  amino acid linker
TVAAP

SEQ ID NO: 164 - protein, 12 aa, Artificial Sequence
  amino acid linker
TVAAPSVFIFPP

SEQ ID NO: 165 - protein, 6 aa, Artificial Sequence
  amino acid linker
QPKAAP

SEQ ID NO: 166 - protein, 13 aa, Artificial Sequence
  amino acid linker
QPKAAPSVTLFPP

SEQ ID NO: 167 - protein, 6 aa, Artificial Sequence
  amino acid linker
AKTTPP

SEQ ID NO: 168 - protein, 13 aa, Artificial Sequence
  amino acid linker
AKTTPPSVTPLAP

SEQ ID NO: 169 - protein, 6 aa, Artificial Sequence
  amino acid linker
AKTTAP

SEQ ID NO: 170 - protein, 13 aa, Artificial Sequence
  amino acid linker
AKTTAPSVYPLAP

SEQ ID NO: 171 - protein, 6 aa, Artificial Sequence
  amino acid linker
ASTKGP

SEQ ID NO: 172 - protein, 13 aa, Artificial Sequence
  amino acid linker
ASTKGPSVFPLAP

SEQ ID NO: 173 - protein, 15 aa, Artificial Sequence
  amino acid linker
GGGGSGGGGSGGGGS

SEQ ID NO: 174 - protein, 15 aa, Artificial Sequence
  amino acid linker
GENKVEYAPALMALS

SEQ ID NO: 175 - protein, 15 aa, Artificial Sequence
  amino acid linker
GPAKELTPLKEAKVS

SEQ ID NO: 176 - protein, 15 aa, Artificial Sequence
  amino acid linker
GHEAAAVMQVQYPAS

SEQ ID NO: 177 - protein, 442 aa, Homo sapiens
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 178 - protein, 212 aa, Homo sapiens
SYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 179 - protein, 239 aa, Artificial Sequence
  protease-cleavable anti-TFPI scFv antibody
EVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSGGGGSLVPRGSGGGGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVL

SEQ ID NO: 180 - DNA, 717 bp, Artificial Sequence
  protease-cleavable anti-TFPI scFv antibody coding sequence
gaagttcagctggtcgaaagtggtggcggtctggttcaaccgggcggttcactgcgtctgtcgtgcgcagcaagcggttttaccttcagttcctatggtatggattgggtccgtcaggcaccgggtaaaggtctggaatgggtgtcatcgattcgtggcagccgcggttctacctattacgccgattcagttaaaggccgttttaccatctctcgcgacaacagtaaaaatacgctgtatctgcagatgaacagcctgcgcgcggaagataccgccgtgtattactgtgcccgtctgtatcgctactggttcgactactggggccagggtacgctggtgaccgttagctctggtggcggtggctctctggttccgcgtggctccggtggcggtggctcatcctatgaactgacccaaccgccgagtgtctccgtgtcaccgggtcagacggcacgtattacctgcagcggtgataacctgccgaaatattacgcgcattggtatcagcaaaaaccgggccaagccccggtggttgtcatcttttatgacgttaatcgtccgtccggtatcccggaacgcttctcgggcagcaactctggtaatacggcaaccctgacgatcagcggcacccaggcaatggatgaagctgactattactgtcaagcatggtggagctctacgccggtgtttggcggtggcacgaaactgaccgtgctg

SEQ ID NO: 181 - protein, 569 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVSASEGRTATEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 182 - protein, 565 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVSASEGRTATVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 183 - protein, 561 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVSASEGRTATGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 184 - protein, 566 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVEGRTATEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 185 - protein, 562 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVEGRTATVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 186 - protein, 558 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLVTVEGRTATGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 187 - protein, 563 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLEGRTATEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 188 - protein, 559 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLEGRTATVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 189 - protein, 555 aa, Artificial sequence
  Synthetic polypeptide
QVELLESGGGLVQPGGSLRLSCAASGFTFSDYYMSWVRQAPGKGLEWVSAISGSGGSTYYADSVKGRFTISRDNSKNTLYLQMNSLRAEDTAVYYCAKRSSSPASDFDYWGQGTLEGRTATGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 190 - protein, 331 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGASEGRTATSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 191 - protein, 327 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGASEGRTATTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 192 - protein, 323 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGASEGRTATSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 193 - protein, 329 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGEGRTATSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194 - protein, 325 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGEGRTATTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 195 - protein, 321 aa, Artificial sequence
  Synthetic polypeptide
QSVLTQPPSASGTPGQRVTISCSGSTSNIGRNAVNWYQQLPGTAPKLLIYGNSNRPSGVPDRFSGSKSGTSASLAISGLRSEDEADYYCAAWDDSLSGWVFGGGTKLTVLGEGRTATSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 196 - protein, 690 aa, Artificial sequence [sequence omitted]

SEQ ID NO: 197 - protein, 683 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKEGRTATSGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 198 - protein, 212 aa, Artificial sequence; identical to SEQ ID NO: 178
  Synthetic polypeptide

SEQ ID NO: 199 - protein, 463 aa, Artificial sequence
  Synthetic polypeptide
AARYWDYDVFGGGTPVGGEGRTATLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 200 - protein, 705 aa, Artificial sequence [sequence omitted]

SEQ ID NO: 201 - protein, 219 aa, Artificial sequence
  Synthetic polypeptide
DIVMTQSPLSLPVTPGEPASISCRSSQSLVFRDGITYLNWYLQKPGQSPQLLIYKGSNRASGVPDRFSGSGSGTDFTLKISRVEAEDVGVYYCQQYDSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 699 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 203 - protein, 700 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSLVPRGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 204 - protein, 717 aa, Artificial sequence [sequence omitted]

SEQ ID NO: 205 - protein, 705 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKEGRTATGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 206 - protein, 710 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKGGGGSLVPRGSGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 207 - protein, 578 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 208 - protein, 334 aa, Artificial sequence
  Synthetic polypeptide
DIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKGGGGSLVPRGSGGGGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 209 - protein, 565 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTLVPRGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 210 - protein, 322 aa, Artificial sequence
  Synthetic polypeptide
DIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELVPRGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 211 - protein, 568 aa, Artificial sequence
  Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTLVPRGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 212 - protein, 330 aa, Artificial sequence
  Synthetic polypeptide
DIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKGGSLVPRGSGGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 213 - protein, 482 aa, Artificial sequence
  Synthetic polypeptide
AARYWDYDVFGGGTPVGGGGGGSLVPRGSGGGGSQVQLQQSGPGLVKPSQTLSLTCAISGDSVSSDSAAWSWIRQSPSRGLEWLGIIYYRSKWYNRYAVSVKSRITINPDTSKNQFSLQLNSVTPEDTAVYYCARWHSDKHWGFDDWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 214 - protein, 476 aa, Artificial sequence
  Synthetic polypeptide
AARYWDYDVFGGGTPVGGGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 215 - protein, 494 aa, Artificial sequence
  Synthetic polypeptide
AARYWDYDVFGGGTPVGGSKATNATLDPRSFLLRNPNDKYEPFWEDEEKNESEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 216 - protein, 482 aa, Artificial sequence
  Synthetic polypeptide
AARYWDYDVFGGGTPVGGEGRTATGGGGSLVPRGSGGGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 217 - protein, 472 aa, Artificial sequence
  Synthetic polypeptide
AARYWDYDVFGGGTPVGGGGRSGSGGRSGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 218 - protein, 463 aa, Artificial sequence
  Synthetic polypeptide
LPHSHRAHSLPPFNPRGPGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 219 - protein, 482 aa, Artificial sequence; identical to SEQ ID NO: 213
  Synthetic polypeptide

SEQ ID NO: 220 - protein, 219 aa, Artificial sequence; identical to SEQ ID NO: 201
  Synthetic polypeptide

SEQ ID NO: 221 - protein, 476 aa, Artificial sequence; identical to SEQ ID NO: 214
  Synthetic polypeptide

SEQ ID NO: 222 - protein, 494 aa, Artificial sequence; identical to SEQ ID NO: 215
  Synthetic polypeptide

SEQ ID NO: 223 - protein, 482 aa, Artificial sequence; identical to SEQ ID NO: 216
  Synthetic polypeptide

SEQ ID NO: 224 - protein, 472 aa, Artificial sequence; identical to SEQ ID NO: 217
  Synthetic polypeptide

SEQ ID NO: 225 - protein, 228 aa, Artificial sequence; identical to SEQ ID NO: 131
  Synthetic polypeptide

SEQ ID NO: 226 - protein, 476 aa, Artificial sequence
  Synthetic polypeptide
AARYWDYDVFGGGTPVGGEGRTATGSLVPRGSGGEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 227 - protein, 473 aa, Artificial sequence
  Synthetic polypeptide
AARYWDYDVFGGGTPVGGEGRTATGSLVPRGSGGLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 228 - protein, 472 aa, Artificial sequence
  Synthetic polypeptide
AARYWDYDVFGGGTPVGGEGRTATLVPRGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 229 - protein, 470 aa, Artificial sequence
  Synthetic polypeptide
AARYWDYDVFGGGTPVGGSLVPRGSGGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 230 - protein, 468 aa, Artificial sequence
  Synthetic polypeptide
AARYWDYDVFGGGTPVGGLVPRGSGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 231 - protein, 468 aa, Artificial sequence
  Synthetic polypeptide
AARYWDYDVFGGGTPVGGEGRTATGSEVQLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 232 - protein, 482 aa, Artificial sequence [sequence omitted]

SEQ ID NO: 233 - protein, 479 aa, Artificial sequence
  Synthetic polypeptide
AARYWDYDVFGGGTPVGGEGRTATGGGGSLVPRGSGGGGSLVESGGGLVQPGGSLRLSCAASGFTFSSYGMDWVRQAPGKGLEWVSSIRGSRGSTYYADSVKGRFTISRDNSKNTLYLQMNSLRAEDTAVYYCARLYRYWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 234 - protein, 442 aa, Artificial sequence; identical to SEQ ID NO: 177
  Synthetic polypeptide

SEQ ID NO: 235 - protein, 246 aa, Artificial sequence
  Synthetic polypeptide
AARYWDYDVFGGGTPVGGEGRTATGSLVPRGSGGSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 236 - protein, 240 aa, Artificial sequence
  Synthetic polypeptide
AARYWDYDVFGGGTPVGGSLVPRGSGGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 237 - protein, 240 aa, Artificial sequence
  Synthetic polypeptide
AARYWDYDVFGGGTPVGGSLVPSGSGGSSYELTQPPSVSVSPGQTARITCSGDNLPKYYAHWYQQKPGQAPVVVIFYDVNRPSGIPERFSGSNSGNTATLTISGTQAMDEADYYCQAWWSSTPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 238 - protein, 54 aa, Artificial sequence
  Synthetic polypeptide
AARYWDYDVFGGGTPVGGEGRTATLVESGGGLVQPGGSLRLSCAASGFTFSSYG

SEQ ID NO: 239 - protein, 63 aa, Artificial sequence
  Synthetic polypeptide
AARDWDFDVFGGGTPVGGGGGGSLVPRGSGGGGSQVQLQQSGPGLVKPSQTLSLTCAISGDSV

SEQ ID NO: 240 - protein, 63 aa, Artificial sequence
  Synthetic polypeptide
AARDWDFDVFGGGTPVGGGGGGSLVPRGSGGGGLVESGGGLVQPGGSLRLSCAASGFTFSSYG

SEQ ID NO: 241 - protein, 82 aa, Artificial sequence
  Synthetic polypeptide
AARDWDFDVFGGGTPVGGSKATNATLDPRSFLLRNPNDKYEPFWEDEEKNESLVESGGGLVQPGGSLRLSCAASGFTFSSYG

SEQ ID NO: 242 - protein, 69 aa, Artificial sequence
  Synthetic polypeptide
AARDWDFDVFGGGTPVGGEGRTATGGGGSLVPRGSGGGGLVESGGGLVQPGGSLRLSCAASGFTFSSYG

SEQ ID NO: 243 - protein, 63 aa, Artificial sequence; identical to SEQ ID NO: 240
  Synthetic polypeptide

SEQ ID NO: 244 - protein, 73 aa, Artificial sequence
  Synthetic polypeptide
AARDWDFDVFGGGTPVGGEGRTATGGGGSLVPRGSGGGGSEVQLVESGGGLVQPGGSLRLSCAASGFTFSSYG

SEQ ID NO: 245 - protein, 67 aa, Artificial sequence
  Synthetic polypeptide
AARDWDFDVFGGGTPVGGEGRTATGSLVPRGSGGEVQLVESGGGLVQPGGSLRLSCAASGFTFSSYG

SEQ ID NO: 246 - protein, 64 aa, Artificial sequence
  Synthetic polypeptide
AARDWDFDVFGGGTPVGGEGRTATGSLVPRGSGGLVESGGGLVQPGGSLRLSCAASGFTFSSYG

SEQ ID NO: 247 - protein, 63 aa, Artificial sequence
  Synthetic polypeptide
AARDWDFDVFGGGTPVGGEGRTATLVPRGSEVQLVESGGGLVQPGGSLRLSCAASGFTFSSYG

SEQ ID NO: 248 - protein, 61 aa, Artificial sequence
  Synthetic polypeptide
AARDWDFDVFGGGTPVGGSLVPRGSGGSEVQLVESGGGLVQPGGSLRLSCAASGFTFSSYG

SEQ ID NO: 249 - protein, 59 aa, Artificial sequence
  Synthetic polypeptide
AARDWDFDVFGGGTPVGGLVPRGSGSEVQLVESGGGLVQPGGSLRLSCAASGFTFSSYG

SEQ ID NO: 250 - protein, 59 aa, Artificial sequence
  Synthetic polypeptide
AARDWDFDVFGGGTPVGGEGRTATGSEVQLVESGGGLVQPGGSLRLSCAASGFTFSSYG

SEQ ID NO: 251 - protein, 73 aa, Artificial sequence
  Synthetic polypeptide
AARDWDFDVFGGGTPVGGEGSTATGGGGSLVPSGSGGGGSEVQLVESGGGLVQPGGSLRLSCAASGFTFSSYG

SEQ ID NO: 252 - protein, 70 aa, Artificial sequence
  Synthetic polypeptide
AARDWDFDVFGGGTPVGGEGRTATGGGGSLVPRGSGGGGSLVESGGGLVQPGGSLRLSCAASGFTFSSYG

SEQ ID NO: 253 - protein, 30 aa, Artificial sequence
  Synthetic polypeptide
DIVMTQSPLSLPVTPGEPASISCRSSQSLV

SEQ ID NO: 254 - protein, 64 aa, Artificial sequence
  Synthetic polypeptide
AARDWDFDVFGGGTPVGGEGRTATGSLVPRGSGGDIVMTQSPLSLPVTPGEPASISCRSSQSLV

SEQ ID NO: 255 - protein, 58 aa, Artificial sequence
  Synthetic polypeptide
AARDWDFDVFGGGTPVGGSLVPRGSGGSDIVMTQSPLSLPVTPGEPASISCRSSQSLV

SEQ ID NO: 256 - protein, 58 aa, Artificial sequence
  Synthetic polypeptide
AARDWDFDVFGGGTPVGGSLVPSGSGGSDIVMTQSPLSLPVTPGEPASISCRSSQSLV

(19) United States
(12) Patent Application Publication
(10) Pub. No.: US 2016/0009817 A1
(43) Pub. Date: Jan. 14, 2016

(54) PRO-DRUG ANTIBODIES AGAINST TISSUE FACTOR PATHWAY INHIBITOR

(71) Applicant: BAYER HEALTHCARE LLC, Whippany, NJ (US)

(72) Inventors: Zhuozhi WANG, Millbrae, CA (US); John MURPHY, Boston, MA (US); Terry HERMISTON, Mill Valley, CA (US); Ying ZHU, Alamo, CA (US); Ruth WINTER, Oakland, CA (US)

(21) Appl. No.: 14/772,373

(22) PCT Filed: Mar. 14, 2014
(86) PCT No.: PCT/US14/29207

§ 371 Date: Sep. 2, 2015

Related U.S. Application Data
(60) Provisional application No. 61/794,024, filed on Mar. 15, 2013.

(51) Int. Cl. C07K 16/36; C07K 16/18

(52) U.S. Cl. CPC C07K 16/36; C07K 16/18; C07K 2317/622; C07K 2317/92; C07K 2317/76; C07K 2317/35; C07K 2319/00

(57) ABSTRACT

This disclosure provides pro-drug antibodies which specifically bind to tissue factor pathway inhibitor (TFPI) only after exposure to proteases from the coagulation cascade. The pro-drug antibodies described are useful for treating bleeding disorders such as hemophilia. When used in such treatments, these pro-drug antibodies show increased half-life relative to other anti-TFPI antibodies, while mitigating the potential for side effects such as thrombosis.

### BACKGROUND

Pursuant to 37 C.F.R. 1.821(c), a sequence listing is submitted herewith as an ASCII compliant text file named “BAYRP0004WO_ST25.txt” created on Mar. 14, 2014 and having a size of ˜312 kilobytes. The content of the aforementioned file is hereby incorporated by reference in its entirety.

This application claims benefit of priority to U.S. Provisional Application Ser. No. 61/794,024, filed Mar. 15, 2013, the entire contents of which are hereby incorporated by reference.

I. Technical Field

The technical field is related to the treatment of hemophilia and other coagulopathies.

II. Related Art

Blood coagulation is a process by which blood forms stable clots to stop bleeding.

The process involves a number of proenzymes and procofactors (or “coagulation factors”) that are circulating in the blood. Those proenzymes and procofactors interact through several pathways through which they are converted, either sequentially or simultaneously, to the activated form. Ultimately, the process results in the activation of prothrombin to thrombin by activated Factor X (FXa) in the presence of Factor Va, ionic calcium, and platelets. The activated thrombin in turn induces platelet aggregation and converts fibrinogen into fibrin, which is then cross linked by activated Factor XIII (FXIIIa) to form a clot.

The process leading to the activation of Factor X can be carried out by two distinct pathways: the contact activation pathway (formerly known as the intrinsic pathway) and the tissue factor pathway (formerly known as the extrinsic pathway). It was previously thought that the coagulation cascade consisted of two pathways of equal importance joined to a common pathway. It is now known that the primary pathway for the initiation of blood coagulation is the tissue factor pathway. Factor X can be activated by tissue factor (TF) in combination with activated Factor VII (FVIIa). The complex of Factor VIIa and its essential cofactor, TF, is a potent initiator of the clotting cascade.

The tissue factor pathway of coagulation is negatively controlled by tissue factor pathway inhibitor (“TFPI”). TFPI is a natural, FXa-dependent feedback inhibitor of the FVIIa/TF complex. It is a member of the multivalent Kunitz-type serine protease inhibitors. Physiologically, TFPI binds to activated Factor X (FXa) to form a heterodimeric complex, which subsequently interacts with the FVIIa/TF complex to inhibit its activity, thus shutting down the tissue factor pathway of coagulation. In principle, blocking TFPI activity can restore FXa and FVIIa/TF activity, thus prolonging the duration of action of the tissue factor pathway and amplifying the generation of FXa, which is the common defect in hemophilia A and B.

Indeed, some preliminary experimental evidence has indicated that blocking the TFPI activity by antibodies against TFPI normalizes the prolonged coagulation time or shortens the bleeding time. For instance, Nordfang et al. showed that the prolonged dilute prothrombin time of hemophilia plasma was normalized after treating the plasma with antibodies to TFPI (Thromb. Haemost., 1991, 66 (4): 464-467). Similarly, Erhardtsen et al. showed that the bleeding time in hemophilia A rabbit model was significantly shortened by anti-TFPI antibodies (Blood Coagulation and Fibrinolysis, 1995, 6: 388-394). These studies suggest that inhibition of TFPI by anti-TFPI antibodies may be useful for the treatment of hemophilia A or B. Only polyclonal anti-TFPI antibody was used in these studies.

Using hybridoma techniques, monoclonal antibodies against recombinant human TFPI (rhTFPI) were prepared and identified. See Yang et al., Chin. Med. J., 1998, 111 (8): 718-721. The effect of the monoclonal antibody on dilute prothrombin time (PT) and activated partial thromboplastin time (APTT) was tested. Experiments showed that anti-TFPI monoclonal antibody shortened dilute thromboplastin coagulation time of Factor IX deficient plasma. It is suggested that the tissue factor pathway plays an important role not only in physiological coagulation but also in hemorrhage of hemophilia (Yang et al., Hunan Yi Ke Da Xue Xue Bao, 1997, 22 (4): 297-300).

U.S. Pat. No. 7,015,194 to Kjalke et al. discloses compositions comprising FVIIa and a TFPI inhibitor, including polyclonal or monoclonal antibodies, or a fragment thereof, for treatment or prophylaxis of bleeding episodes or coagulative treatment. The use of such composition to reduce clotting time in normal mammalian plasma is also disclosed. It is further suggested that a Factor VIII or a variant thereof may be included in the disclosed composition of FVIIa and TFPI inhibitor. A combination of FVIII or Factor IX with TFPI monoclonal antibody is not suggested. In addition to the treatment for hemophilia, it has also been suggested that TFPI inhibitors, including polyclonal or monoclonal antibodies, can be used for cancer treatment (see U.S. Pat. No. 5,902,582 to Hung).

Accordingly, improved antibodies specific for TFPI are needed for treating hematological diseases and cancer.

### SUMMARY OF THE DISCLOSURE

Thus, in accordance with the present disclosure, there is provided an antibody comprising (a) a first variable domain comprising a first light and a first heavy chain variable region, the first variable domain binding immunologically to Tissue Factor Pathway Inhibitor (TFPI); (b) a masking domain linked to the amino terminus of the first light and/or first heavy chain variable region; and (c) a protease cleavable linker interposed between the first light and/or first heavy chain variable region and the masking domain. The protease cleavable domain may be a thrombin, plasmin, Factor VIIa or Factor Xa cleavage site. The masking domain may comprise a second variable domain comprising a second light and a second heavy chain variable region. The antibody may be an IgG1, an IgG2, an IgG3, an IgG4, an IgM, an IgA 1, an IgA2, a secretory IgA, an IgD, and an IgE antibody. The antibody may be a human or humanized antibody, and/or a single-chain antibody. The antibody may be bivalent and comprise two masking domains, one linked to the amino terminus of each first light chain variable region, or bivalent and comprise two masking domains, one linked to the amino terminus of each first heavy chain variable region, or bivalent and comprise four masking domains, one linked to the amino terminus of each first light chain variable region and each first heavy chain variable region, such as where two of the masking domains are a second light chain variable region, and two of the masking domains are a second heavy chain variable region, wherein the second light and heavy chain variable regions form a second variable domain. The second variable domain may bind to tissue factor (TF), a red blood cell, or albumin. The masking domain may be albumin binding protein. The antibody may bind to Kunitz domain 2 of human tissue factor pathway inhibitor.

Also provided is an expression vector comprising a coding region for an antibody as described above under the control of a promoter, and a cell comprising such an expression vector. Also provided is a pharmaceutical formulation comprising an antibody as described above formulated with a pharmaceutically acceptable buffer, carrier or diluent.

In another embodiment, there is provided a method of treating a coagulation disorder in a subject comprising administering to the subject an antibody comprising (a) a first variable domain comprising a first light and a first heavy chain variable region, the first variable domain binding immunologically to Tissue Factor Pathway Inhibitor (TFPI); (b) a masking domain linked to the amino terminus of the first light and/or first heavy chain variable region; and (c) a protease cleavable linker interposed between the first light and/or first heavy chain variable region and the masking domain, in an amount effective to promote coagulation in the subject. The protease cleavable domain may be a thrombin, plasmin, Factor VIIa or Factor Xa cleavage site. The masking domain may comprise a second variable domain comprising a second light and a second heavy chain variable region. The antibody may be an IgG1, an IgG2, an IgG3, an IgG4, an IgM, an IgA 1, an IgA2, a secretory IgA, an IgD, and an IgE antibody. The antibody may be a human or humanized antibody, and/or a single-chain antibody. The antibody may be bivalent and comprise two masking domains, one linked to the amino terminus of each first light chain variable region, or bivalent and comprise two masking domains, one linked to the amino terminus of each first heavy chain variable region, or bivalent and comprise four masking domains, one linked to the amino terminus of each first light chain variable region and each first heavy chain variable region, such as where two of the masking domains are a second light chain variable region, and two of the masking domains are a second heavy chain variable region, wherein the second light and heavy chain variable regions form a second variable domain. The second variable domain may bind to tissue factor (TF), a red blood cell, or albumin. The masking domain may be albumin binding protein. The subject may be a human or a non-human mammal. The subject may suffer from trauma, hemophilia (e.g., hemophilia A or B) or cancer. The antibody may be administered systemically, or administered locally or regionally to a site of bleeding. The antibody may be administered subcutaneously, intravenously or intra-arterially. The antibody may bind to Kunitz domain 2 of human tissue factor pathway inhibitor.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Illustration of an embodiment of an anti-TFPI pro-drug antibody and how it functions in vivo.

FIG. 2. Illustration of the potential masking strategies of an anti-TFPI pro-drug antibody.

FIGS. 3A-B. Vector map of TF-binding prodrug where the variable regions against TFPI and TF were tandem linked. (FIG. 3A) Vector map of HC1-pTTF5 gA200 anti-TFPI pro-drug antibody fragment. (FIG. 3B) Vector map of LC1-pTTF 641 anti-TFPI pro-drug antibody fragment.

FIGS. 4A-C. Vector map of TF-binding prodrug and RBC-binding prodrug where the scFv against TF or RBC was linked on amino-terminus of heavy chain of anti-TFPI antibody. (FIG. 4A) Vector map of pQM1-3E1 Osc-gA200HC anti-TFPI pro-drug antibody fragment. (FIG. 4B) Vector map of pQM1-T119sc-gA200HC anti-TFPI pro-drug antibody fragment. (FIG. 4C) Vector map of pQM1/gA200LC anti-TFPI pro-drug antibody fragment.

FIGS. 5A-B. Vector map of albumin-biding prodrug. (FIG. 5A) Vector map of pQM1-56E4-gA200H anti-TFPI pro-drug antibody fragment. (FIG. 5B) Vector map of pQM1-56E4-gA200L anti-TFPI pro-drug antibody fragment.

FIG. 6. SDS-PAGE of 3E10-scFv-gA200 and Ter119-scFv-gA200 anti-TFPI IgG with Coomassie staining and in non-reducing condition without dithiothreitol (DTT) and reducing condition with DTT.

FIG. 7. Graph of TFPI binding ELISA to determine the binding affinity of 56E4-gA200 relative to native gA200.

FIG. 8. Graph of Ter119sc-gA200 binding to RBCs as a function of antibody concentration.

FIG. 9. Graph of results of BIACORE™ measurement for different anti-TFPI pro-drug antibodies and unmodified anti-TFPI antibody, gA200, relative binding percentage to TFPI in the presence or absence of human serum albumin (HSA).

FIGS. 10A-G. (FIG. 10A) Graph of peak thrombin as a function of antibody concentration for anti-TFPI pro-drug antibody, 56E4-gA200, and anti-TFPI antibody, gA200. (FIG. 10B) Graph of thrombin generation as a function of different concentrations of anti-TFPI pro-drug antibody, 56E4-gA200, and anti-TFPI antibody, gA200, showing the concentration of thrombin produced at each antibody concentration. (FIG. 10C) Comparison of thrombin generation profiles of prodrug TPP-2654, which can be activated both by thrombin and FXa activation, with its parental antibody gA200. The ability of thrombin to activate TPP-2654 was assessed by adding exogenous thrombin, then hirudin to inactivate the thrombin added. Controls include reactions where buffer was added in place of thrombin. (FIG. 10D) Titration of thrombin needed to activate prodrug TPP-2654 is shown. The thrombin concentrations tested are in the range potentially achievable physiologically. (FIG. 10E) The ability of FXa to activate prodrug TPP-2654 was assessed indirectly. FXa and thrombin levels were increased by increasing TF concentration used to initiate the TGA reaction. By comparing the ability of exogenous thrombin to enhance TPP-2654 responses with those achieved by both FXa and thrombin activation, the relative contribution of FXa to activate prodrug can be gauged. (FIG. 10F) Thrombin generation of a prodrug TPP-2652, activated by thrombin alone, is shown. Here titration studies indicated that the prodrug TPP-2652 required ˜2.5 U/mL thrombin to convert to active TFPI Ab. (FIG. 10G) The relative insensitivity of TPP-2652 to FXa can be observed in the results of the TF titration experiment. In contrast to TPP-2654 which was designed to be activated by both FXa and thrombin activation, TPP-2652 showed a lesser increase in thrombin generation at the higher TF dose used (compare FIG. 10G with FIG. 10E).

FIG. 11. Graph of the effect of varying concentration of albumin on anti-TFPI pro-drug antibody and unmodified anti-TFPI antibody.

FIG. 12. Sequences of heavy and light chains that can be combined to prepare anti-TFPI pro-drug antibodies according to the present disclosure.

FIG. 13. Sequences of heavy and light chains of anti-TFPI pro-drug antibodies according to the present disclosure.

FIGS. 14A-B. Amino terminal sequences of selected heavy (FIG. 14A) and heavy and light chains (FIG. 14B) of anti-TFPI pro-drug antibodies according to the present disclosure.

FIG. 15A. TFPI-binding of albumin-binding anti-TFPI prodrug in the absence or presence of human or monkey albumin (surface plasmon resonance-Biacore data);

FIG. 15B. TFPI-binding of albumin-binding anti-TFPI prodrug, after treated with or without thrombin or FXa, in the absence or presence of human/monkey albumin (surface plasmon resonance).

FIGS. 16A-C. (FIGS. 16A-B) Mass-spectrum of anti-TFPI prodrug TPP-2652 and TPP-2654 after thrombin cleavage. (FIG. 16C) Mass-spectrum of anti-TFPI prodrug TPP-2654 cleaved with Fxa.

### DETAILED DESCRIPTION OF THE DISCLOSURE

This disclosure describes a safe and long-acting antibody against Tissue Factor Pathway Inhibitor (TFPI) for hemophilia and other therapies. Currently, anti-TFPI antibodies are in preclinical and clinical development, respectively, but the in vivo half-life of anti-TFPI antibodies is relatively shorter than that of a other IgG antibodies. This is likely due to target-mediated clearance. Additionally, concern has also been raised that anti-TFPI antibody may cause side effects, in a patient with inflammation or treated with FVIIa.

To address these issues, the anti-TFPI pro-drug antibodies described in this disclosure have been developed. These antibodies have significantly reduced binding to TFPI before they are exposed to protease(s) generated from coagulation cascade. Once the coagulation is initiated and the protease(s) generated, the proteases activate the anti-TFPI antibody by cleaving the masking domain thus increasing its binding on TFPI. These pro-drug antibodies can be used to treat bleeding disorders such as hemophilia, while offering better safety and pharmacokinetics profile as compared to previously described anti-TFPI antibodies.

### 1. Anti-TFPI Pro-drug Antibodies

The antibodies disclosed herein specifically bind to TFPI; i.e., they bind to TFPI with an affinity that is higher (e.g., at least two-fold higher) than their binding affinity for an irrelevant antigen (e.g., BSA, casein). The term “tissue factor pathway inhibitor” or “TFPI” as used herein refers to any variant, isoform and species homolog of human TFPI that is naturally expressed by cells.

In some embodiments, pro-drug antibodies bind to TFPI with an affinity of at least about 10⁵ M⁻¹ to about 10¹² M⁻¹ (e.g., 10⁵ M⁻¹, 10^{5.5} M⁻¹, 10⁶ M⁻¹, 10^{6.5} M⁻¹, 10⁷ M⁻¹, 10^{7.5} M⁻¹, 10⁸ M⁻¹, 10^{8.5} M⁻¹, 10⁹ M⁻¹, 10^{9.5} M⁻¹, 10¹⁰ M⁻¹, 10^{10.5} M⁻¹, 10¹¹ M⁻¹, 10^{11.5} M⁻¹, 10¹² M⁻¹). The affinity (Ka) of antibody binding to an antigen can be assayed using any method known in the art including, for example, immunoassays such as enzyme-linked immununospecific assay (ELISA), Bimolecular Interaction Analysis (BIA) (e.g., Sjolander & Urbaniczky; Anal. Chem. 63:2338-2345, 1991; Szabo, et al., Curr. Opin. Struct. Biol. 5:699-705, 1995, both of which are incorporated herein by reference), and fluorescence-activated cell sorting (FACS) for quantification of antibody binding to cells that express an antigen. BIA is a technology for analyzing biospecific interactions in real time, without labeling any of the interactants (e.g., BIACORE™). Changes in the optical phenomenon surface plasmon resonance (SPR) can be used as an indication of real-time reactions between biological molecules.

An anti-TFPI pro-drug antibody can be constructed using a substantially full-length immunoglobulin molecule (e.g., IgG1, IgG2a, IgG2b, IgG3, IgG4, IgM, IgD, IgE, IgA), an antigen binding fragment thereof, such as a Fab or F(ab′)2, or a construct containing an antigen binding site, such as a scFv, Fv, or diabody, which is capable of specific binding to TFPI. The term “antibody” also includes other protein scaffolds that are able to orient antibody complementarity-determining region (CDR) inserts into the same active binding conformation as that found in natural antibodies such that the binding to TFPI observed with these chimeric proteins is maintained relative to the TFPI binding activity of the natural antibody from which the CDRs were derived.

An “isolated antibody” as used herein is an antibody which is substantially free of other antibodies having different antigenic specificities (e.g., an isolated antibody that binds to TFPI is substantially free of antibodies that bind antigens other than TFPI). An isolated antibody that binds to an epitope, isoform, or variant of human TFPI may, however, have cross-reactivity to other related antigens, e.g., from other species (e.g., TFPI species homologs). An isolated antibody can be substantially free of other cellular material and/or chemicals.

Particular anti-TFPI antibodies are disclosed in U.S. Patent Publications US 2012/20268917, US2012/0108796, US2011/0229476 and International Patent Publication WO2012/135671, the entire disclosure of each of these documents being incorporated by reference herein.

A. Masked Antibodies

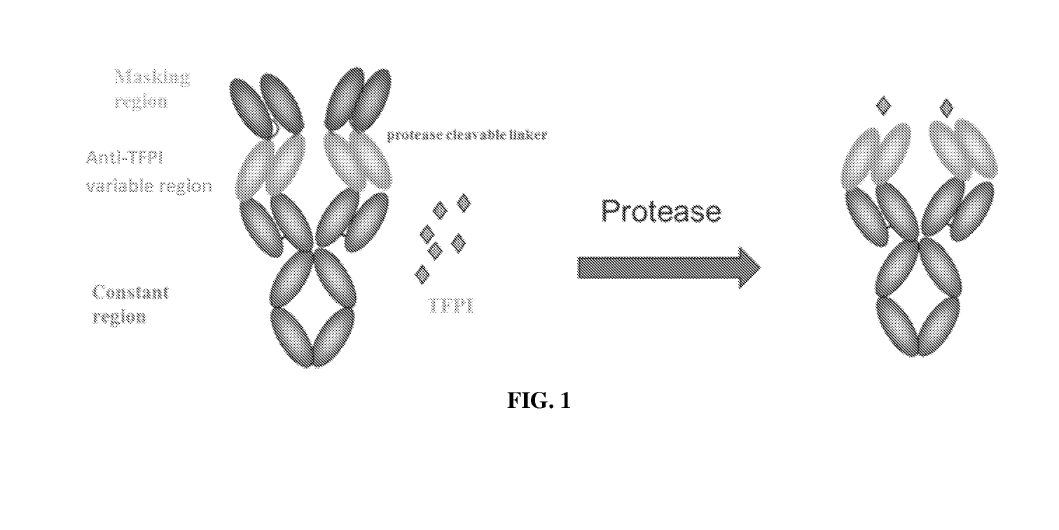

In some embodiments, the pro-drug antibodies disclosed herein are engineered to have a masking domain which reduces ability of the antibodies to bind to TFPI. These masking domains could recognize an element of the coagulation cascade or other related markers. In some embodiments, the masking domain includes the following elements which recognize biological molecules such as tissue factor (TF), red blood cells (RBCs), and/or albumin. These masking domains are attached to the variable region of the antibody through a protease cleavage site as shown in FIG. 1. These masking domains could be an antibody, peptide, protein, or another scaffold. Regardless, the masking domains prevent the binding of the antibody to TFPI through its variable region until removed.

The pro-drug antibodies disclosed herein are engineered to comprise a protease cleavage site recognized by one or more proteases, the cleavage of which will release the masking domain and permit the antibody to bind to TFPI. As used herein, “protease cleavage site” refers to an amino acid sequence that is recognized and cleaved by a protease. In some embodiments, the protease cleavage site is positioned to mask the variable region of an anti-TFPI antibody and is shown in FIG. 1. In some embodiments, anti-TFPI pro-drug antibodies include one or more protease cleavage sites that can be cleaved by thrombin, plasmin, and/or Factor Xa. It is also envisioned that other protease cleavage sites for proteases activated or upregulated by the coagulation cascade could be used. In some embodiments, the amino acid sequence masking the variable region of an anti-TFPI pro-drug antibody comprises a polypeptide linker in addition to the protease cleavage site (as illustrated, for example, in FIG. 1) and/or an antibody, peptide, protein, or another scaffold which binds to TF, RBCs, or albumin. The linker can be a single amino acid or a polypeptide sequence (e.g., up to 100 amino acids). For example, the linker can be GGGGS (SEQ ID NO: 149). Other useful linkers include those shown in SEQ ID NOS: 151-176. In other embodiments, no linker is present, and the cleavage site itself is inserted on the variable region in such a manner as to mask its binding to TFPI as shown in FIG. 1 with the antibody, peptide, protein, or another scaffold which binds to TF, RBCs, or albumin.

At least two optimal cleavage sites for thrombin have been determined: (1) X₁-X₂—P—R—X₃-X₄ (SEQ ID NO: 147), where X₁ and X₂ are hydrophobic amino acids and X₃ and X₄ are nonacidic amino acids; and (2) GRG. Thrombin specifically cleaves after the arginine residue. Plasmin can also cleave the two aforementioned cleavage sites, however with less specificity as compared to thrombin. Other useful thrombin cleavage sites are provided as SEQ ID NOS: 1-60. Other useful plasmin cleavages sites are provided as SEQ ID NOS: 12, 47, 48, 53, and 61-130. In some embodiments, the cleavage site is LVPRGS (SEQ ID NO: 137).

In some embodiments, a Factor Xa cleavage site, such as I-(E or D)-G-R (SEQ ID NO: 148), is used. Other useful Factor Xa cleavage sites are provided as SEQ ID NOS: 29, 59, and 61-69.

In addition to cleavage site, a second binding site of protease, so-called exosite, can be introduced into a anti-TFPI prodrug to make the cleavage more efficient. The exosite of thrombin can be from the native exosite of protease substrates or inhibitor, such as PAR1, fibrinogen and hirudin. The exosite can also be a derivative of other exosite from proteins.

B. Antibody Synthesis

Anti-TFPI pro-drug antibodies can be produced synthetically or recombinantly. A number of technologies are available to produce antibodies. For example, phage-antibody technology can be used to generate antibodies (Knappik et al., J. Mol. Biol. 296:57-86, 2000, which is incorporated herein by reference). Another approach for obtaining antibodies is to screen a DNA library from B cells as described in WO 91/17271 and WO 92/01047, both of which are incorporated herein by reference. In these methods, libraries of phage are produced in which members display different antibodies on their outer surfaces. Antibodies are usually displayed as Fv or Fab fragments. Phage displaying antibodies are selected by affinity enrichment for binding to a selected protein. Antibodies can also be produced using trioma methodology (e.g., Oestberg et al., Hybridoma 2:361-367, 1983; U.S. Pat. No. 4,634,664; U.S. Pat. No. 4,634,666, all of which are incorporated herein by reference).

Antibodies can also be purified from any cell that expresses the antibodies, including host cells that have been transfected with antibody-encoding expression constructs. The host cells can be cultured under conditions whereby the antibodies are expressed. Purified antibody can be separated from other cellular components that can associate with the antibody in the cell, such as certain proteins, carbohydrates, or lipids, using methods well known in the art. Such methods include, but are not limited to, size exclusion chromatography, ammonium sulfate fractionation, ion exchange chromatography, affinity chromatography, and preparative gel electrophoresis. Purity of the preparations can be assessed by any means known in the art, such as SDS-polyacrylamide gel electrophoresis. A preparation of purified antibodies can contain more than one type of antibody.

Alternatively, anti-TFPI pro-drug antibodies can be produced using chemical methods to synthesize its amino acid sequence, such as by direct peptide synthesis using solid-phase techniques (e.g., Merrifield, J. Am. Chem. Soc. 85:2149-2154, 1963; Roberge et al., Science 269:202-204, 1995, both of which are incorporated herein by reference). Protein synthesis can be performed using manual techniques or by automation. Optionally, fragments of antibodies can be separately synthesized and combined using chemical methods to produce a full-length molecule.

In some embodiments, an anti-TFPI pro-drug antibody can also be constructed in a “single chain Fv (scFv) format,” in which a protease cleavage site is inserted in or around a peptide linker, antibody, peptide, protein, or another scaffold on the variable region in such a manner as to mask its ability to recognize TFPI. As the peptide linker is necessary to hold together the two variable regions of a scFv for antigen binding, cleavage of the peptide linker or flanking region allows a protease of interest to inactivate or to down-regulate the binding of scFv to its antigen.

In some embodiments, anti-TFPI pro-drug antibodies are constructed in “IgG format,” having two binding sites, and can comprise one, two, three, or four protease cleavage sites between the variable region and an antibody, peptide, protein, or another scaffold in such a manner as to mask its ability to recognize TFPI. In each case, a protease cleavage site can be flanked on either or both sides by a linker. Further, in each case, the cleavage sites can be the same or different.

### 2. Polynucleotides

This disclosure also provides polynucleotides encoding pro-drug antibodies. These polynucleotides can be used, for example, to produce quantities of the antibodies for therapeutic use.

Antibody-encoding cDNA molecules can be made with standard molecular biology techniques, using mRNA as a template. Thereafter, cDNA molecules can be replicated using molecular biology techniques known in the art and disclosed in manuals such as Sambrook, et al., (Molecular Cloning: A Laboratory Manual, (Second Edition, Cold Spring Harbor Laboratory Press; Cold Spring Harbor, N.Y.; 1989) Vol. 1-3, which is incorporated herein by reference). An amplification technique, such as PCR, can be used to obtain additional copies of the polynucleotides. Alternatively, synthetic chemistry techniques can be used to synthesize polynucleotides encoding anti-TFPI pro-drug antibodies.

To express a polynucleotide encoding an antibody, the polynucleotide can be inserted into an expression vector that contains the necessary elements for the transcription and translation of the inserted coding sequence. Methods that are well known to those skilled in the art can be used to construct expression vectors containing sequences encoding antibodies and appropriate transcriptional and translational control elements. These methods include in vitro recombinant DNA techniques, synthetic techniques, and in vivo genetic recombination. Such techniques are described, for example, in Sambrook, et al. (1989) and in Ausubel, et al., (Current Protocols in Molecular Biology, John Wiley & Sons, New York, N. Y., 1995), both of which are incorporated herein by reference.

A variety of expression vector/host systems can be utilized to contain and express sequences encoding antibodies. These include, but are not limited to, microorganisms, such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV); or bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in strength and specificity. Depending on the vector system and host, any number of suitable transcription and translation elements, including constitutive and inducible promoters, can be used. For example, when cloning in bacterial systems, inducible promoters can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses can be used. If it is necessary to generate a cell line that contains multiple copies of a nucleotide sequence encoding an antibody, vectors based on SV40 or EBV can be used with an appropriate selectable marker.

### 3. Methods of Preparing Antibodies To TFPI

General texts describing additional useful molecular biological techniques, including the preparation of antibodies, are Berger and Kimmel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, a joint venture between Green Publishing Associates, Inc. and John Wiley & Sons, Inc. (supplemented through 2000)); Harlow et al., (Monoclonal Antibodies: A Laboratory Manual, Cold Spring Harbor Laboratory Press (1988), Paul [Ed.]); Fundamental Immunology, (Lippincott Williams & Wilkins (1998)); and Harlow, et al. (Using Antibodies: A Laboratory Manual, Cold Spring Harbor Laboratory Press (1998)) all of which are incorporated herein by reference.

Therapeutic antibodies for human diseases have been generated using genetic engineering to create murine, chimeric, humanized or fully human antibodies. Murine monoclonal antibodies were shown to have limited use as therapeutic agents because of a short serum half-life, an inability to trigger human effector functions, and the production of human antimouse-antibodies. Brekke and Sandlie, “Therapeutic Antibodies for Human Diseases at the Dawn of the Twenty-first Century,” Nature 2, 53, 52-62 (January 2003) Chimeric antibodies have been shown to give rise to human anti-chimeric antibody responses. Humanized antibodies further minimize the mouse component of antibodies. However, a fully human antibody avoids the immunogenicity associated with murine elements completely. In particular, chronic prophylactic treatment such as would be required for hemophilia treatment with an anti-TFPI monoclonal antibody has a high risk of development of an immune response to the therapy if an antibody with a murine component or murine origin is used due to the frequent dosing required and the long duration of therapy. For example, antibody therapy for hemophilia A may require weekly dosing for the lifetime of a patient. This would be a continual challenge to the immune system.

Thus, the need exists for a fully human antibody for antibody therapy for hemophilia and related genetic and acquired deficiencies or defects in coagulation.

Therapeutic antibodies have been made through hybridoma technology described by Koehler and Milstein in “Continuous Cultures of Fused Cells Secreting Antibody of Predefined Specificity,” Nature 256, 495-497 (1975). Fully human antibodies may also be made recombinantly in prokaryotes and eukaryotes. Recombinant production of an antibody in a host cell rather than hybridoma production is preferred for a therapeutic antibody. Recombinant production has the advantages of greater product consistency, likely higher production level, and a controlled manufacture that minimizes or eliminates the presence of animal-derived proteins. For these reasons, it may be desirable to have a recombinantly produced monoclonal anti-TFPI antibody.

The monoclonal antibody may be produced recombinantly by expressing a nucleotide sequence encoding the variable regions of the monoclonal antibody according to the embodiments of the invention in a host cell. With the aid of an expression vector, a nucleic acid containing the nucleotide sequence may be transfected and expressed in a host cell suitable for the production. Accordingly, also provided is a method for producing a monoclonal antibody that binds with human TFPI comprising: (a) transfecting a nucleic acid molecule encoding a monoclonal antibody of the invention into a host cell, (b) culturing the host cell so to express the monoclonal antibody in the host cell, and optionally (c) isolating and purifying the produced monoclonal antibody, wherein the nucleic acid molecule comprises a nucleotide sequence encoding a monoclonal antibody of the present invention.

In one example, to express the antibodies, or antibody fragments thereof, DNAs encoding partial or full-length light and heavy chains obtained by standard molecular biology techniques are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain encoding genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or .beta.-globin promoter.

In addition to the antibody chain genes and regulatory sequences, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Examples of selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Examples of mammalian host cells for expressing the recombinant antibodies include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells, HKB11 cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods, such as ultrafiltration, size exclusion chromatography, ion exchange chromatography and centrifugation.

### 4. Use of Partial Antibody Sequences To Express Intact Antibodies

Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998, Nature 332:323-327; Jones et al., 1986, Nature 321:522-525; and Queen et al., 1989, Proc. Natl. Acad. Sci. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. It is not necessary to obtain the entire DNA sequence of a particular antibody in order to recreate an intact recombinant antibody having binding properties similar to those of the original antibody (see WO 99/45962). Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. For this reason, it is necessary to use the corresponding germline leader sequence for expression constructs. To add missing sequences, cloned cDNA sequences can be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts are used to design an overlapping set of synthetic oligonucleotides to create synthetic V sequences with identical amino acid coding capacities as the natural sequences. The synthetic heavy and kappa chain sequences can differ from the natural sequences in three ways: strings of repeated nucleotide bases are interrupted to facilitate oligonucleotide synthesis and PCR amplification; optimal translation initiation sites are incorporated according to Kozak's rules (Kozak, 1991, J. Biol. Chem. 266:19867-19870); and HindIII sites are engineered upstream of the translation initiation sites.

For both the heavy and light chain variable regions, the optimized coding, and corresponding non-coding, strand sequences are broken down into 30-50 nucleotide sections at approximately the midpoint of the corresponding non-coding oligonucleotide. Thus, for each chain, the oligonucleotides can be assembled into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCR products. These overlapping products are then combined by PCR amplification to form the complete variable region. It may also be desirable to include an overlapping fragment of the heavy or light chain constant region in the PCR amplification to generate fragments that can easily be cloned into the expression vector constructs.

The reconstructed heavy and light chain variable regions are then combined with cloned promoter, translation initiation, constant region, 3′ untranslated, polyadenylation, and transcription termination sequences to form expression vector constructs. The heavy and light chain expression constructs can be combined into a single vector, co-transfected, serially transfected, or separately transfected into host cells which are then fused to form a host cell expressing both chains.

Thus, in another aspect, the structural features of a human anti-TFPI antibody, e.g., TP2A8, TP2G6, TP2G7, TP4B7, etc., are used to create structurally related human anti-TFPI antibodies that retain the function of binding to TFPI. More specifically, one or more CDRs of the specifically identified heavy and light chain regions of the monoclonal antibodies of the invention can be combined recombinantly with known human framework regions and CDRs to create additional, recombinantly-engineered, human anti-TFPI antibodies of the invention.

### 5. Pharmaceutical Compositions

An anti-TFPI pro-drug antibody can be provided in a pharmaceutical composition comprising a pharmaceutically acceptable carrier. The pharmaceutically acceptable carrier preferably is non-pyrogenic. A pharmaceutical composition comprising an anti-TFPI pro-drug antibody can be administered alone or in combination with at least one other agent, such as stabilizing compound, which can be administered in any sterile, biocompatible pharmaceutical carrier, including, but not limited to, saline, buffered saline, dextrose, and water. A variety of aqueous carriers can be employed, e.g., 0.4% saline, 0.3% glycine, and the like. These solutions are sterile and generally free of particulate matter. These solutions can be sterilized by conventional, well known sterilization techniques (e.g., filtration). The compositions can contain pharmaceutically acceptable auxiliary substances as required to approximate physiological conditions such as pH adjusting and buffering agents, etc. The concentration of anti-TFPI pro-drug antibody in a pharmaceutical composition can vary widely, i.e., from less than about 0.5%, usually at or at least about 1% to as much as 15 or 20% by weight and will be selected primarily based on fluid volumes, viscosities, etc., according to the particular mode of administration selected. See U.S. Pat. No. 5,851,525, which is incorporated herein by reference, for example. If desired, more than one different anti-TFPI pro-drug antibody can be included in a pharmaceutical composition.

In addition to the active ingredients, pharmaceutical compositions can contain suitable pharmaceutically-acceptable carriers comprising excipients and auxiliaries that facilitate processing of the compositions into preparations which can be used pharmaceutically. Pharmaceutical compositions can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means.

After pharmaceutical compositions have been prepared, they can be placed in an appropriate container and labeled for treatment of an indicated condition. Such labeling would include amount, frequency, and method of administration. The compositions may further be packaged in kits containing one or more containers held together by suitable packaging material including molded Styrofoam and plastic blow-molded containers, optionally including instructions for storage and use.

### 6. Methods of Treating Bleeding Disorders

A. Disorders

Hemophilia is a group of hereditary genetic disorders that impair the body's ability to control blood clotting or coagulation, which is used to stop bleeding when a blood vessel is broken. Hemophilia A (clotting factor VIII deficiency) is the most common form of the disorder, present in about 1 in 5,000-10,000 male births. Hemophilia B (factor IX deficiency) occurs in around 1 in about 20,000-34,000 male births.

Like most recessive sex-linked, X chromosome disorders, haemophilia is more likely to occur in males than females. This is because females have two X chromosomes while males have only one, so the defective gene is guaranteed to manifest in any male who carries it. Because females have two X chromosomes and haemophilia is rare, the chance of a female having two defective copies of the gene is very remote, so females are almost exclusively asymptomatic carriers of the disorder. Female carriers can inherit the defective gene from either their mother or father, or it may be a new mutation. Although it is not impossible for a female to have haemophilia, it is unusual: a female with haemophilia A or B would have to be the daughter of both a male haemophiliac and a female carrier, while the non-sex-linked haemophilia C due to coagulant factor XI deficiency, which can affect either sex, is more common in Jews of Ashkenazi (east European) descent but rare in other population groups.

Haemophilia lowers blood plasma clotting factor levels of the coagulation factors needed for a normal clotting process. Thus, when a blood vessel is injured, a temporary scab does form, but the missing coagulation factors prevent fibrin formation, which is necessary to maintain the blood clot. A haemophiliac does not bleed more intensely than a person without it, but can bleed for a much longer time. In severe haemophiliacs even a minor injury can result in blood loss lasting days or weeks, or even never healing completely. In areas such as the brain or inside joints, this can be fatal or permanently debilitating.

Other bleeding disorders that can be treated by antibodies of the present disclosure include acquired platelet function defects, congenital platelet function defects, congenital protein C or S deficiency, disseminated intravascular coagulation (DIC), Factor II deficiency, Factor V deficiency, Factor VII deficiency, Factor X deficiency, Factor XII deficiency, idiopathic thrombocytopenic purpura (ITP), and Von Willebrand's disease.

B. Pharmaceutical Compositions, Routes and Dosages

Pharmaceutical compositions comprising one or more anti-TFPI pro-drug antibodies can be administered to a patient alone, or in combination with other agents, drugs or coagulation factors, to treat hemophilia or other clotting disorders. A “therapeutically effective dose” of an anti-TFPI pro-drug antibody refers to that amount of anti-TFPI pro-drug antibody that will promote coagulation or reduce bleeding time. The determination of a therapeutically effective dose is well within the capability of those skilled in the art.

A therapeutically effective dose can be estimated initially either in cell culture assays or in animal models, usually rats, mice, rabbits, dogs, or pigs. An animal model also can be used to determine the appropriate concentration range and route of administration. Such information can then be used to determine useful doses and routes for administration in humans.

Therapeutic efficacy and toxicity, e.g., ED50 (the dose therapeutically effective in 50% of the population) and LD50 (the dose lethal to 50% of the population) of an anti-TFPI pro-drug antibody can be determined by standard pharmaceutical procedures in cell cultures or experimental animals. The dose ratio of toxic to therapeutic effects is the therapeutic index, and it can be expressed as the ratio, LD50/ED50.

Pharmaceutical compositions that exhibit large therapeutic indices are preferred. Data obtained from cell culture assays and animal studies are used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the patient who requires treatment. Dosage and administration are adjusted to provide sufficient levels of the anti-TFPI pro-drug antibody or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

In some embodiments, therapeutically effective in vivo dosages of an anti-TFPI pro-drug antibody are in the range of about 5 μg to about 100 mg/kg, about 1 mg to about 50 mg/kg, about 10 mg to about 50 mg/kg of patient body weight.

The mode of administration of a pharmaceutical composition comprising an anti-TFPI pro-drug antibody can be any suitable route which delivers the antibody to the host (e.g., subcutaneous, intramuscular, intravenous, or intranasal administration).

In some embodiments, an anti-TFPI pro-drug antibody is administered without other therapeutic agents. In some embodiments, an anti-TFPI pro-drug antibody is administered in combination with other agents, such as drugs or coagulation factors, to enhance initial production of thrombin while ensuring that the thrombin level stays below the range that may cause thrombosis in some people with coagulopathy. The administration of the anti-TFPI pro-drug antibody can be before, after, or at substantially the same time as the administration of other agents.

### 7. EXAMPLES

The following examples are included to further illustrate various aspects of the disclosure. It should be appreciated by those of skill in the art that the techniques which follow represent techniques and/or compositions discovered by the inventor to function well in the practice of specific embodiments, and may constitute preferred modes for its practice. However, those of skill in the art will understand, in light of the present disclosure, that changes can be made in these embodiments and still obtain a like or similar result without departing from the spirit and scope of the disclosure.

### Example 1

### Anti-TFP1 Pro-Drug Antibody Design Strategy

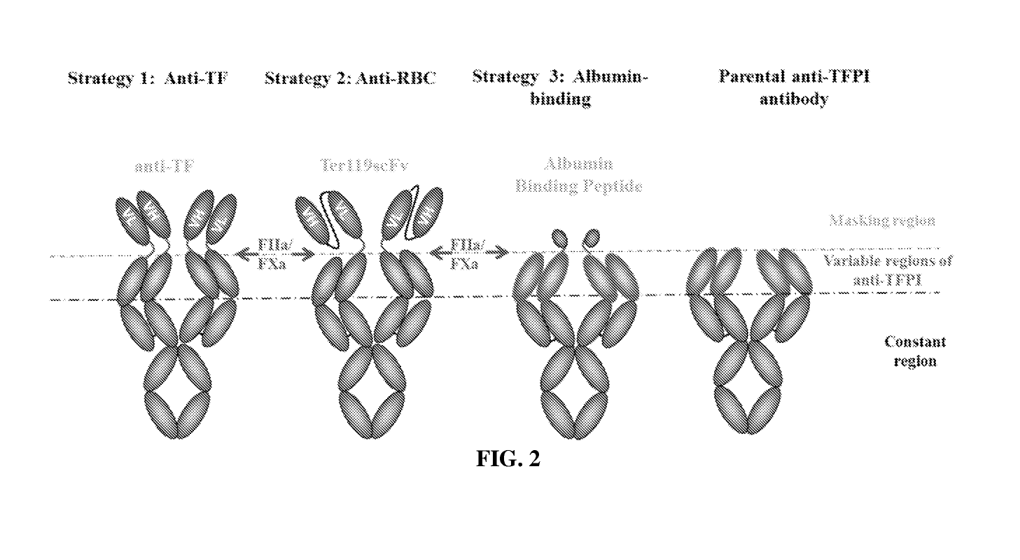

To mask the anti-TFPI activity, at least three strategies have been envisioned, however additional strategies are contemplated. Anti-tissue factor antibody domains, anti-erythrocyte antibody domains or an albumin binding peptide can be used as the masking domain. The masking function may involve the pro-drug antibody binding to the first target, such as tissue factor, red blood cells or albumin. When anti-TFPI pro-drug antibodies are in their latent form, there will be no binding or significantly reduced binding on TFPI until the masking regions are cleaved by a protease generated from the coagulation cascade, as shown in the FIG. 1. Different formats of pro-drug antibodies have been designed. Although all the formats comprise Fc region, which allows FcRn binding to provide long half-life, the variable region can be modified, including tandem-linked variable region, scFv—variable region fusion, peptide-variable region fusion etc. Representations of these pro-drug antibodies are shown in FIG. 2. The parental antibodies of the current envisioned pro-drug antibodies were discovered from human antibody libraries. These antibodies have been extensively optimized to improve their affinity and functionality. The parental antibodies, gA200 and gB9.7, have high affinity and specificity to human TFPI, promoting tissue factor (TF) initiated coagulation.

### Masking Methodologies

A. Tissue Factor Binding

Tissue factor (TF) is a protein present in subendothelial tissue and leukocytes necessary for the initiation of thrombin formation from the zymogen prothrombin. Tissue factor is only exposed to the blood stream thus initiating clotting when an injury occurs. Therefore, targeting TF allows the anti-TFPI pro-drug antibody activated on the injury site. The masking domain of TF-binding incorporated into the anti-TFPI pro-drug antibody could be an TF-binding antibody, peptide, or an alternative scaffold that do not block the function of TF.

B. Anti-erythrocyte Binding

RBCs (red blood cells) have been used as carrier or depot for delivery of drugs and enzymes. RBCs are biocompatible, biodegradable, posse long circulation half-life and can be loaded with variety of biologically active substances. Surface modification with antibodies has been shown to improve their target specificity and to increase their circulation half-life. In this disclosure, an anti-RBC antibody was used as the masking domain fused on the N-terminus of anti-TFPI antibody. This anti-TFPI pro-drug antibody results in a pro-drug with a longer potential circulation time than that of unmodified parental anti-TFPI antibody. Binding of the pro-drug on RBCs will further decrease its ability to bind TFPI until the masking domain has been cleaved.

C. Albumin-Binding Peptide

Albumin has emerged as a versatile carrier for therapeutic and diagnostic agents, primarily for diagnosing and treating diabetes, cancer, rheumatoid arthritis and infectious diseases. Human serum albumin is the most abundant protein in the body with a concentration in circulation of approximately 40 mg/mL. Albumin has molecular weight of 67 kDa. An albumin-binding moiety can be used as masking domain fused on the N-terminus of anti-TFPI antibody, resulting in an anti-TFPI pro-drug antibody with potential longer circulation time than that of parental anti-TFPI antibody. Although an albumin-binding peptide was used in the pro-drug construct, the albumin-binding moiety can be a peptide, a natural albumin-binding domain, a scaffold, an antibody or antibody fragment, such as Fab, scFv, domain antibody and other derivatives.

D. Selection of Proteases and Design of Cleavage Sites

When injury occurs, tissue factor (TF) becomes exposed to the blood stream and activates Factor VII to form a TF/FVIIa complex. The TF/FVIIa complex consequently activates Factor X and FXa activate prothrombin to thrombin, causing fibrin formation and blood clotting. The main role of the tissue factor pathway is to generate a “thrombin burst,” a process by which thrombin, the most important constituent of the coagulation cascade in terms of its feedback activation roles, is released instantaneously. In addition, a series of other coagulation factors are activated in the coagulation cascade:
TF-FVIIa activates FIX and FX.
FVII is itself activated by thrombin, FXIa, FXII and FXa.
FXa and its co-factor FVa form the prothrombinase complex, which activates prothrombin to thrombin.
Thrombin then activates other components of the coagulation cascade, including FV and FVIII) activation releases FVIII from being bound to vWF. Finally thrombin activates FXI, which, in turn, activates FIX
FVIIIa is the co-factor of FIXa, and together they form the “tenase” complex, which activates FX; and so the cycle continues. (“Tenase” is a contraction of “ten” and the suffix “-ase” used for enzymes.)

Many of the aforementioned proteases, such as FVIIa, FXa and thrombin, can be used to activate anti-TFPI pro-drug antibodies. In this disclosure, the cleavage sites of FXa and thrombin were designed into the masking domain, but it is envisioned that any of the other proteases mentioned could also be incorporated into an anti-TFPI pro-drug antibody.

### Example 2

### Vector Maps of Pro-Drug Antibodies

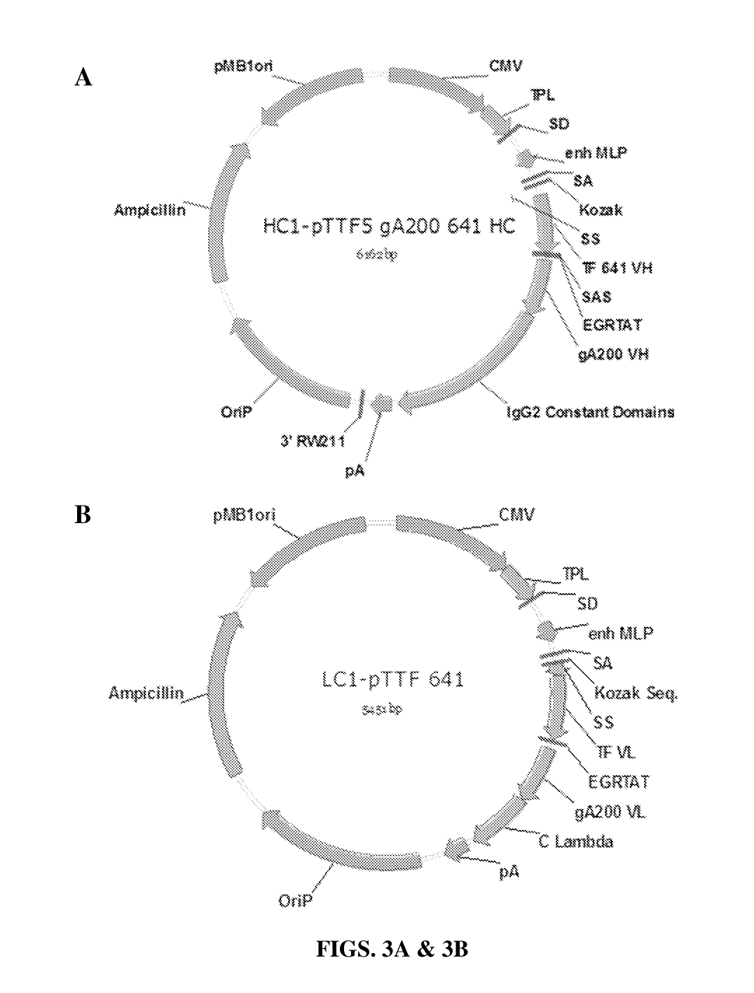

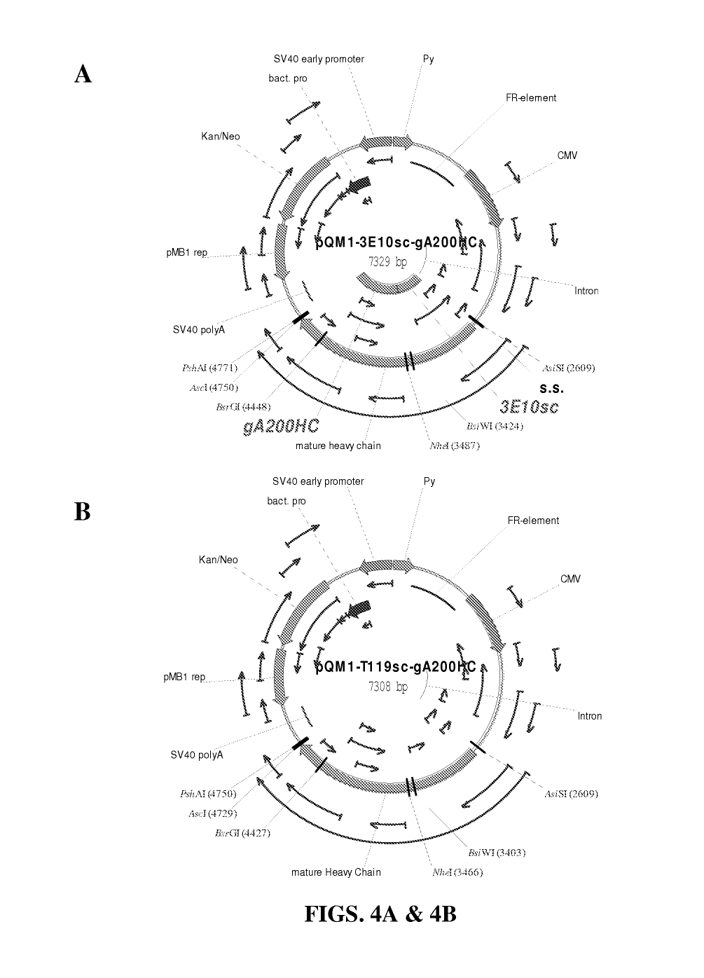

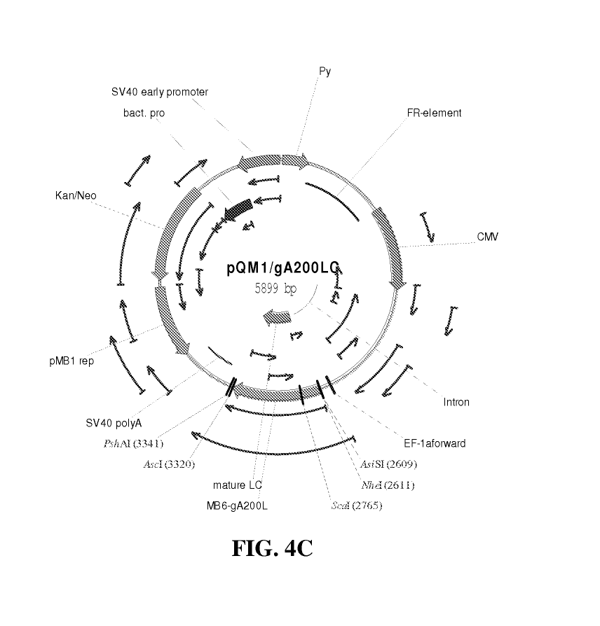

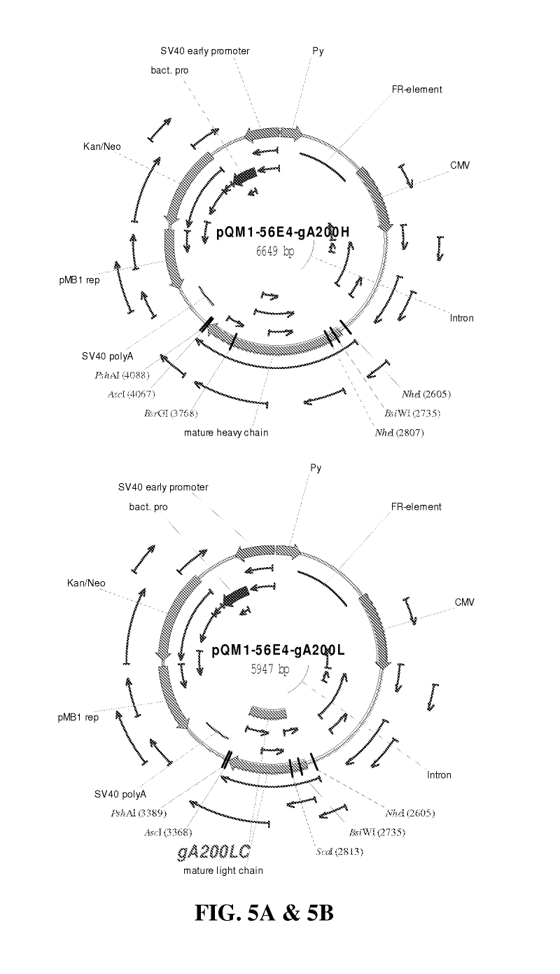

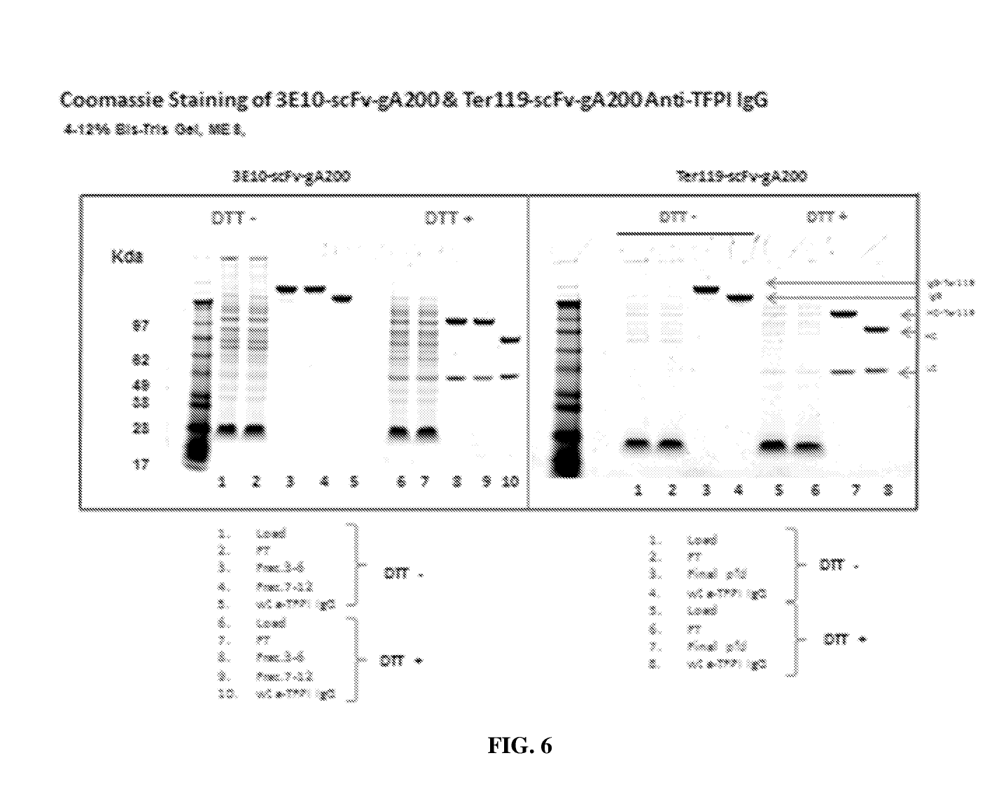

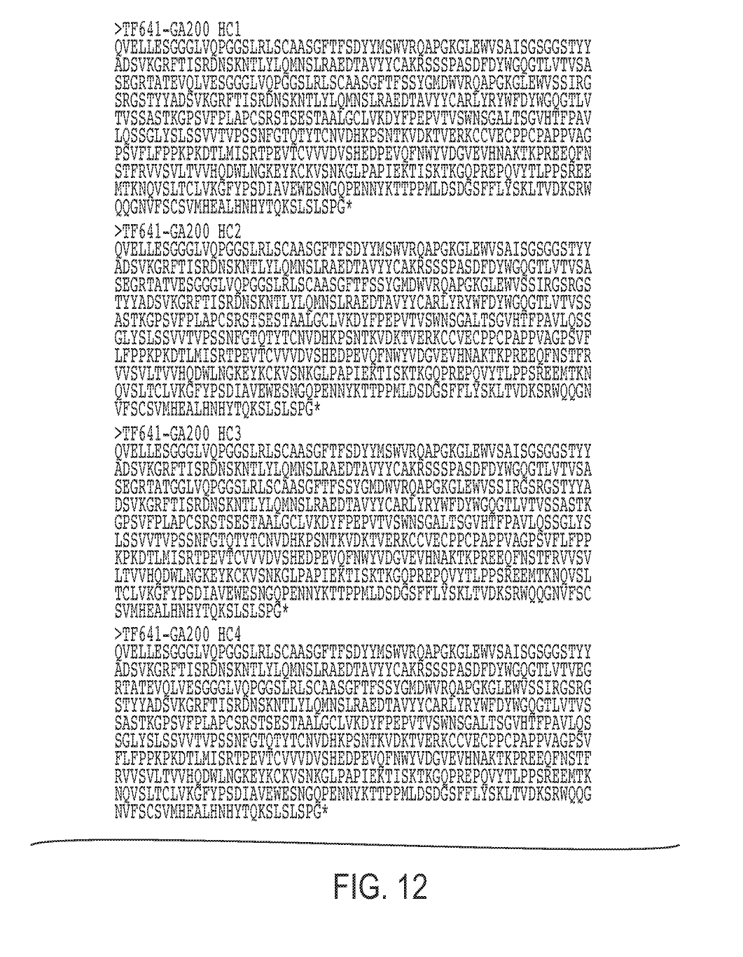

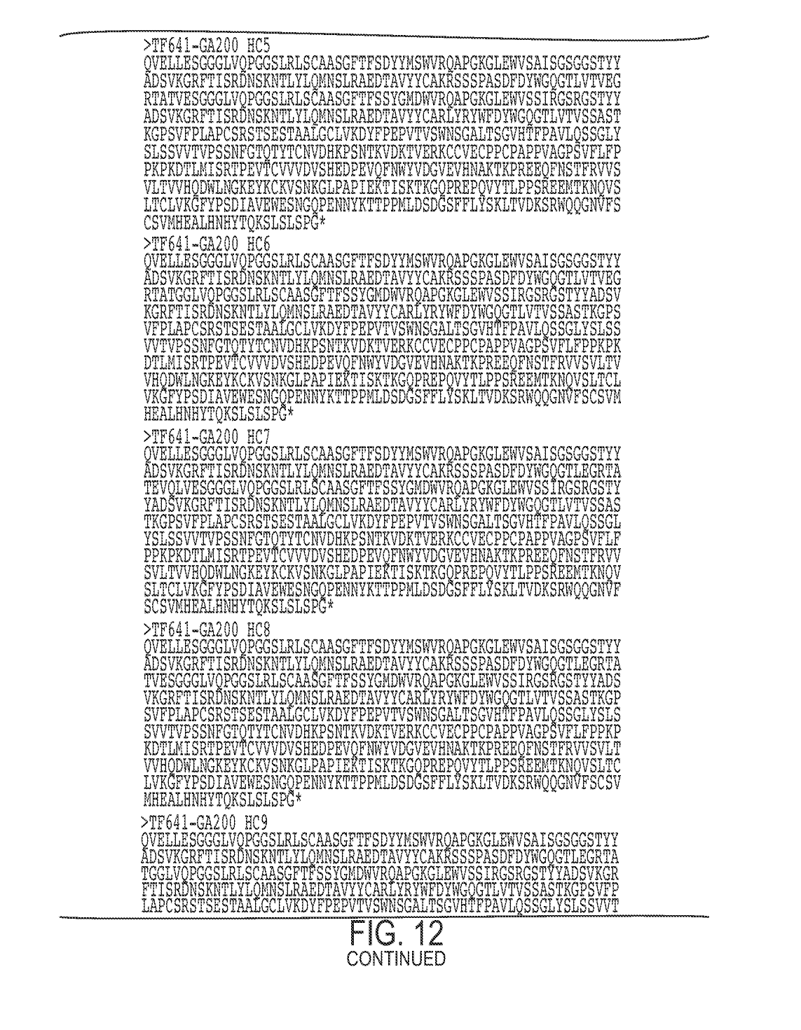

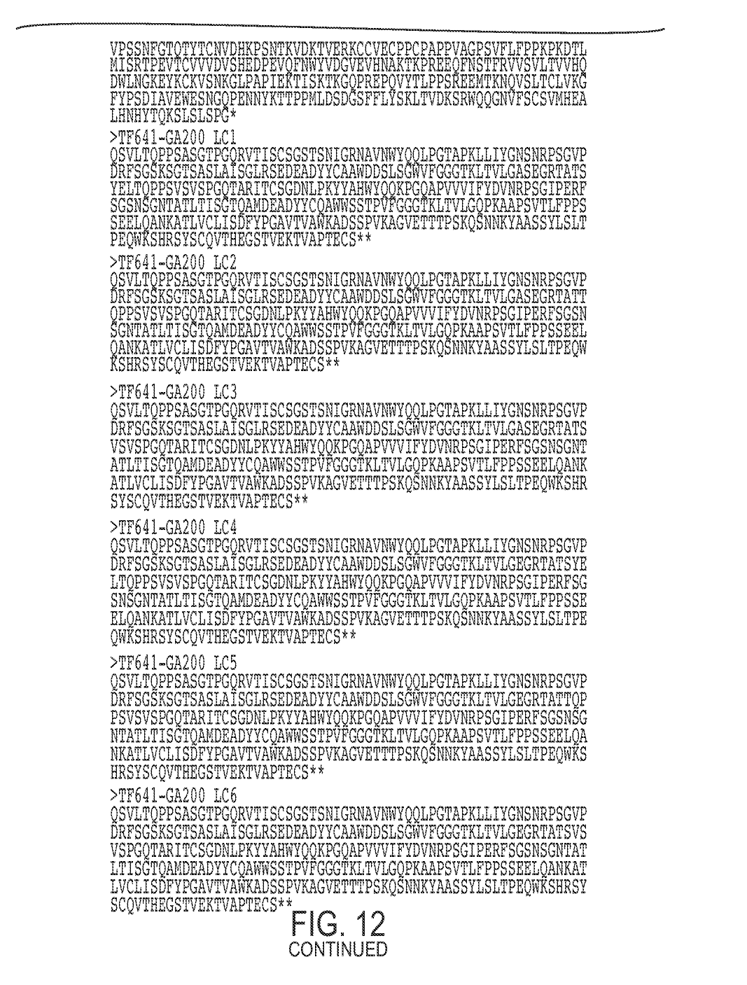

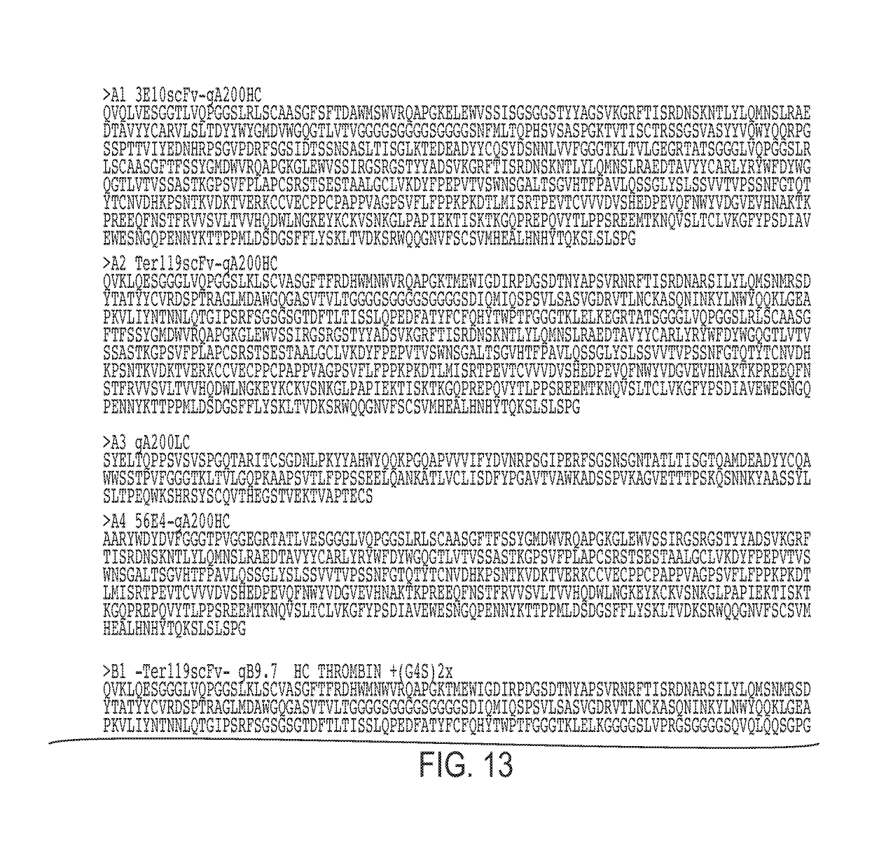

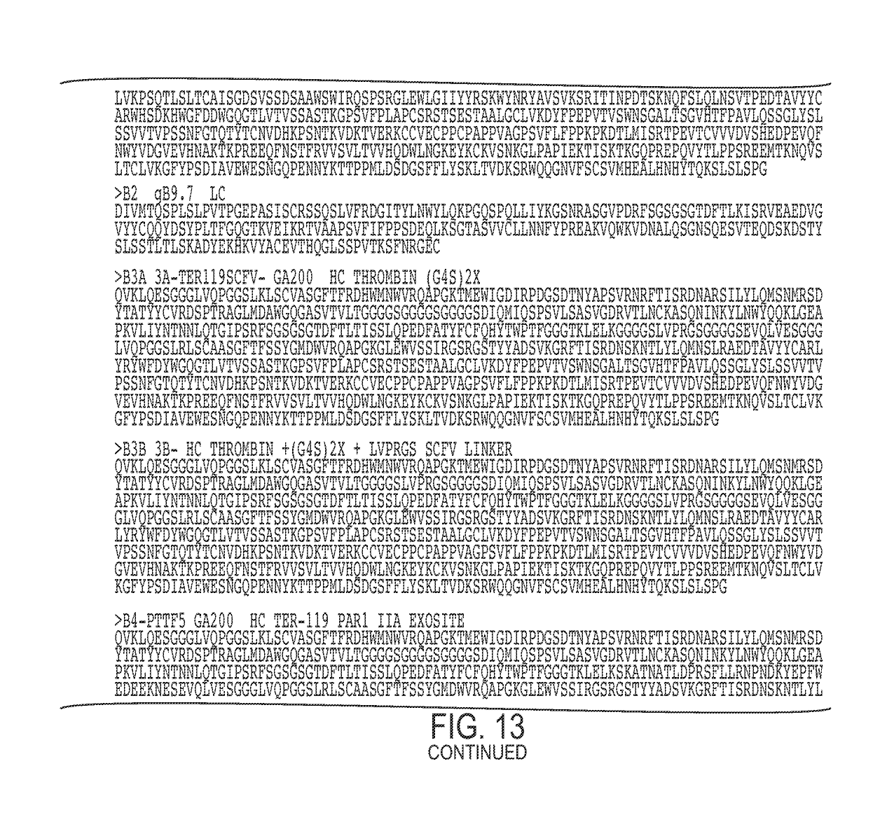

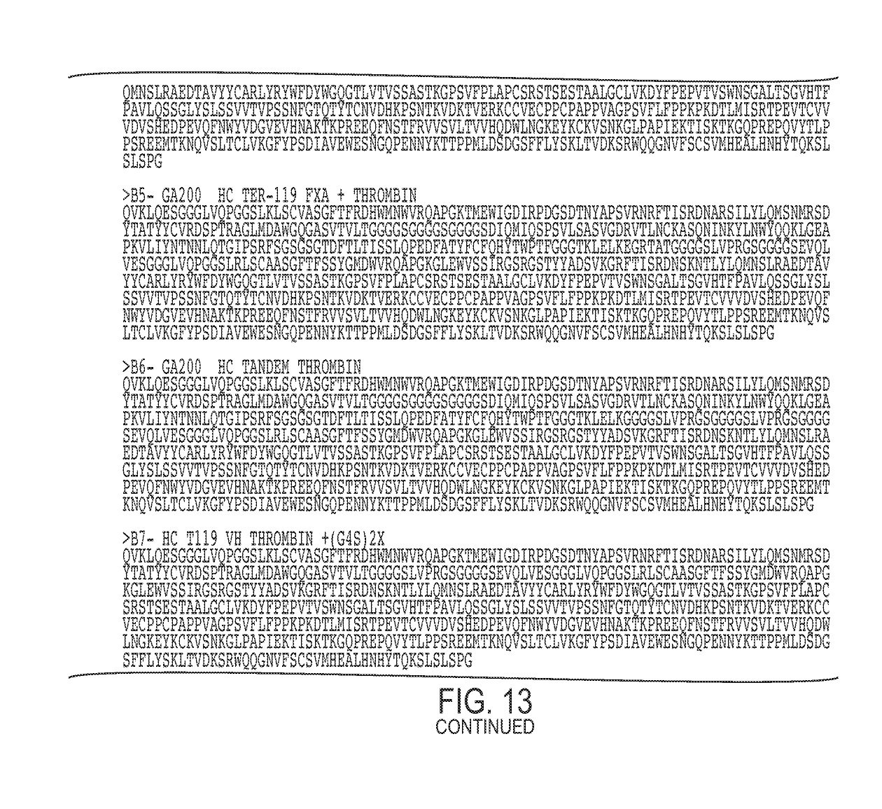

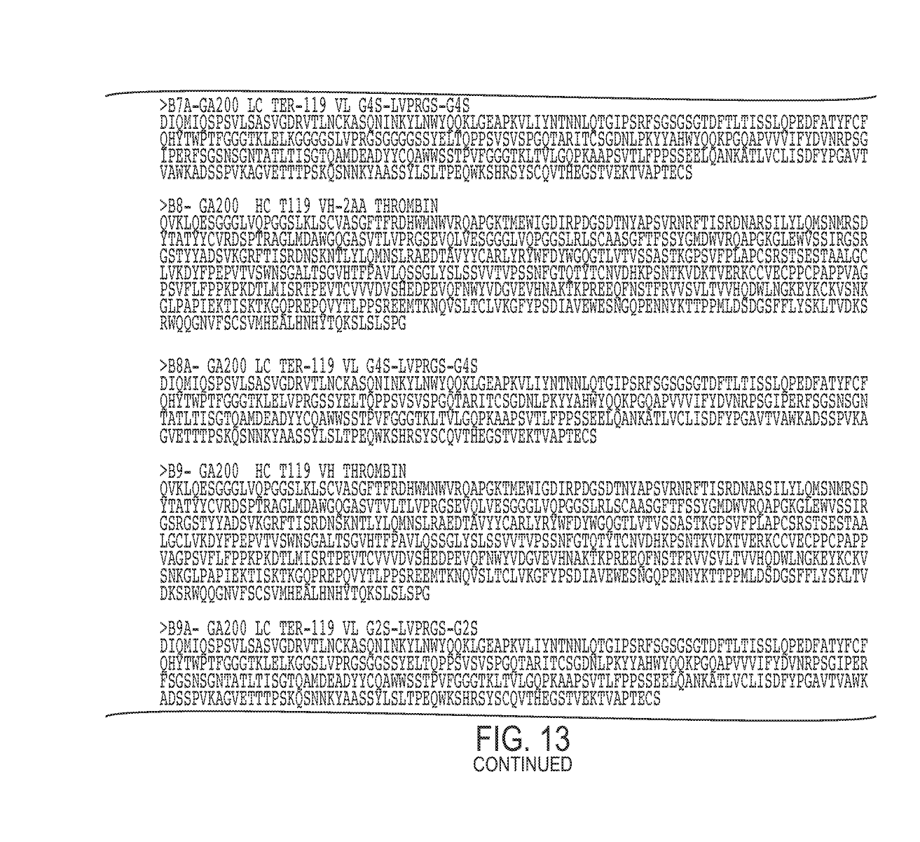

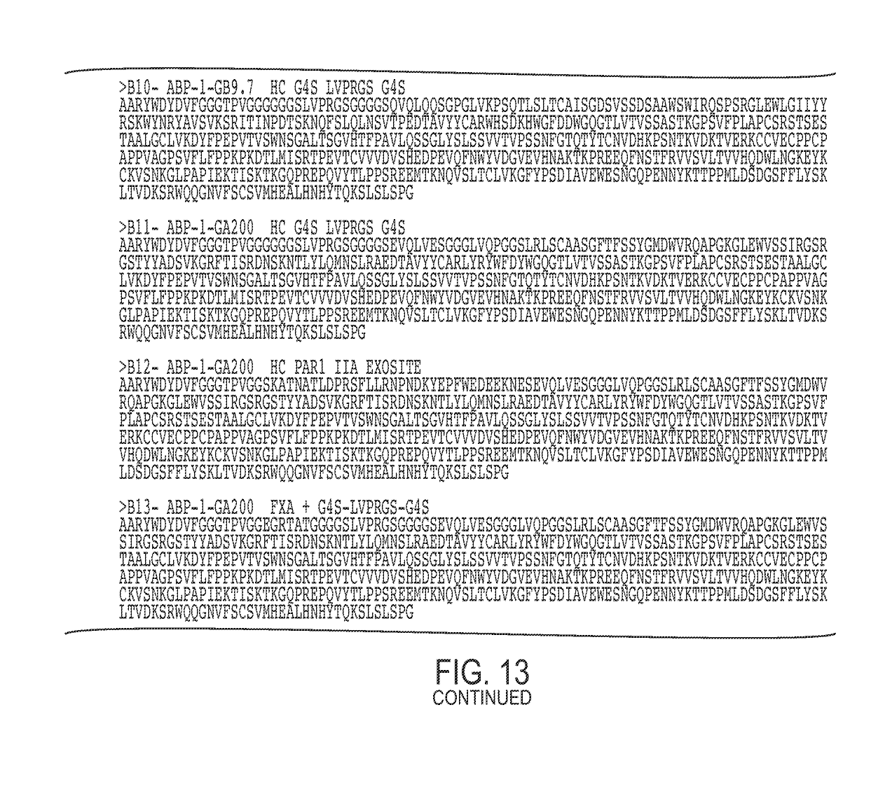

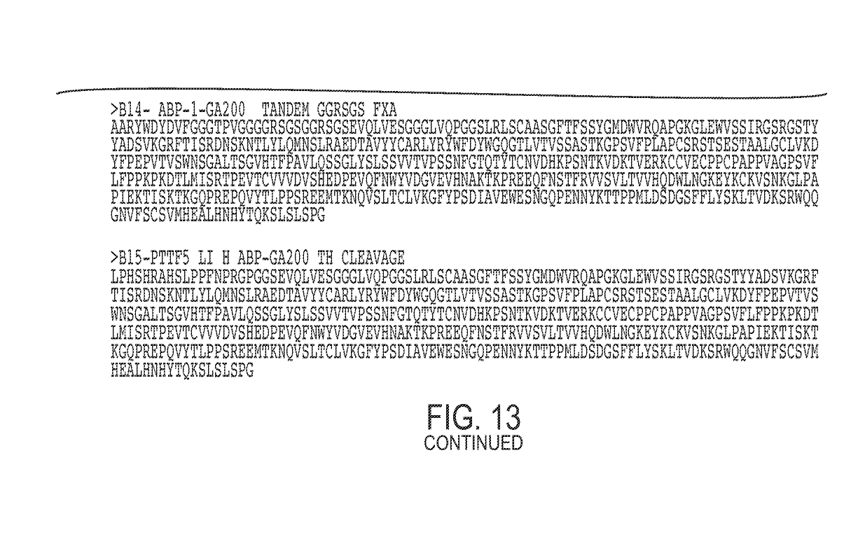

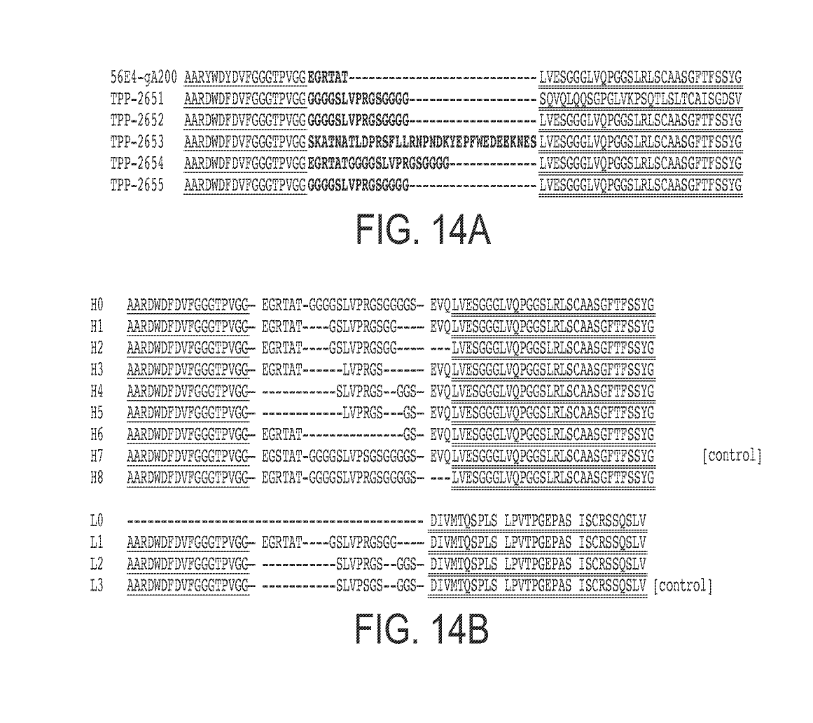

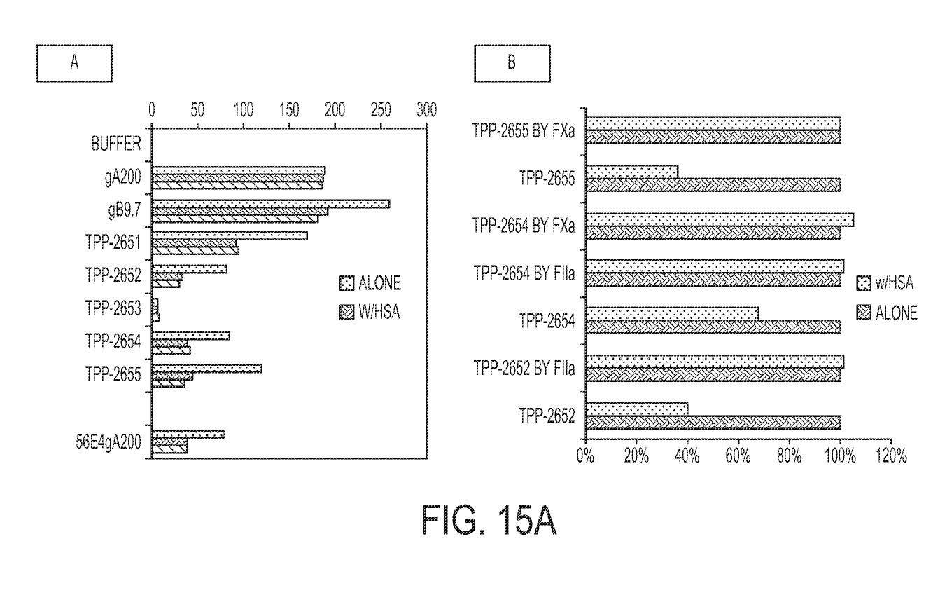

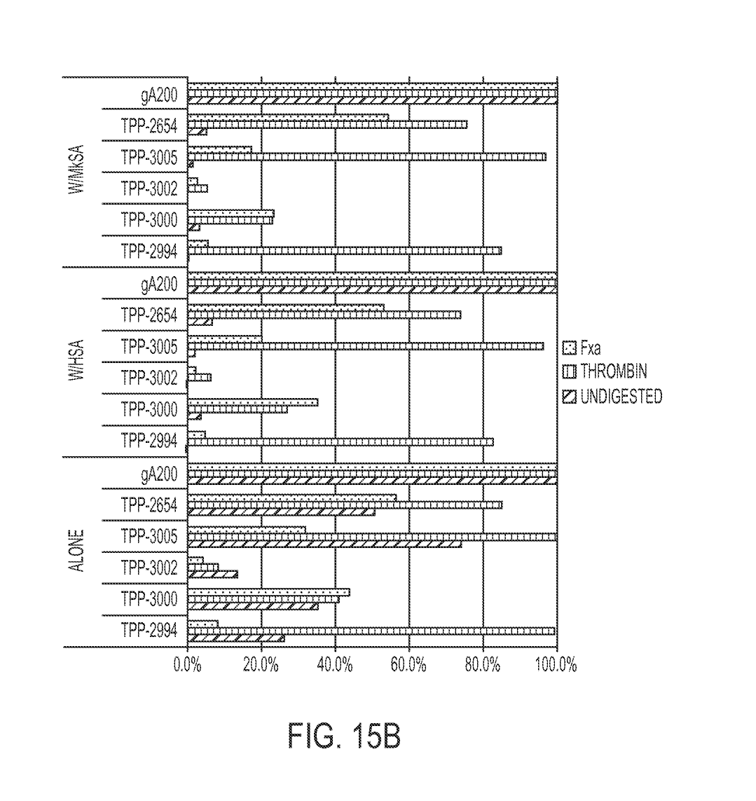

Nine gA200-ProDrug heavy chains (HC) and six gA200 light chain variants (LC) were constructed using Infusion cloning (Clontech) into the expression plasmid pTTF5. All variants contained the FXa six amino acid cleavage site EGRTAT. The muteins contained various N-terminal and C-terminal deletions flanking the cleavage site. A representative plasmid map of HC1 mutein and LC1 mutein is shown in FIGS. 3A-B. The DNA sequence of the 15 light and heavy chain variants are shown in FIG. 12. The HC muteins are named HC1 through HC9, the LC muteins are named LC1 through LC6. The representative plasmid vector map of the scFv (anti-RBC or anti-TF) fused with anti-TFPI are shown in FIGS. 4A-4C and the representative plasmid vector map of albumin binding peptide fused with anti-TFPI are shown in FIGS. 5A-B. FIGS. 13-14 provide the sequences for 15 constructs comprising various light and heavy chain combinations with engineered cleavage sites.

### Example 3

### Expression and Production of Pro-Drug Antibodies

When Chinese hamster ovary cells were used as host cells, transfection was performed using Neucleofection technology from Amaxa. Briefly, 2×10⁶ cells per reaction were pelleted at 1000 rpm for 5 minutes. Pellets were re-suspended in 100 μl of Nucleofector solution V per reaction. Two μg of pQM1-3E10sc-gA200HC with pQM1-gA200LC, pQM1-Ter119sc-gA200HC with pQM1-gA200LC, or pQM1-56E4-gA200HC with pQM1-56E4-gA200LC were added to the cells respectively. The DNA/cells in solution V were then transferred to the Nucleocuvette vessels. Electroporation was performed in the Nucleofector® using program U024. After electroporation, 0.5 mL of warmed medium was added to the cells immediately, then transferred to 6-well plates with 4.5 mL per well of pre-warmed Qmix 1 medium (without antibiotics), and put back to 37° C. incubator on shaker. The expression of pro-drug antibodies was measured 3-4 days post transfection. For positively expressing cells, stable pool was generated. The cells were diluted to 0.5×10⁶/mL, and G418 was added to 0.7 mg/mL. Once the cell density reached 3-4×10⁶/mL, the cells were diluted again to 0.4×10⁶/mL and maintained in Qmix 1 containing 0.7 mg/mL of G418 all time. The selection took approximately two weeks, followed by a production stage. When cell viability recovered to >95%, and cell density reached 3.5-4×10⁶/mL, the culture temperature was switched to 30° C. The conditioned media were harvested 4-7 days after temperature switching. The cells were removed by centrifugation at 5000 rpm for 30 minutes. The conditioned media were concentrated 5× using a Millipore concentrator, followed an additional centrifugation at 9000 rpm for 40 minutes.

When HEK293-6E cells were used as the host cells, they were maintained in F17 medium supplemented with 4 mM L-glutamine, 0.1% Pluronic F68, and 25 mg/L G418 as suspension culture. Transfection was performed using Polyethylenimine (PEI, 251(D, linear). Briefly, 1×10⁶ cells/ml were inoculated the day before transfection. On the day of Transfection, adjusted cell density to 1.7×10⁶/ml. To transfect 1 L of cells, 0.5 mg of each VEC-4581 and VEC-4568 (for TPP2651), or VEC-4583 and 4568 (for TPP2654) were diluted in 500 ml F17 medium, and 2 ml of PEI (PEI stock at 1 mg/ml) diluted in 500 ml of F17. Combine the diluted DNA and PEI, and add to cells after 10′ incubation at room temperature. Cells were then put back to 37° C. incubator on shaker with 125 rpm. 24 h post Transfection, feed the cells with 1% ultra-low IgG FBS, and 0.5 mM Valproic acid. The expression of pro-drug antibodies was measured 3-4 days post Transfection, and the expression was terminated when cell viability dropped down to 70%. The conditioned medium was then harvested by centrifugation at 2000 rpm for 10 minutes to remove the cells, and followed by an additional centrifugation at 9000 rpm for 40 minutes.

### Example 4

### Purification of Anti-TFPI Pro-Drug Antibody

Pro-drug proteins were purified from CHO cell conditioned media using a MabSelect Protein A column (5 mL HiTrap, GE HealthCare, #28-4082-55). Media was either concentrated 5 to 10-fold by ultrafiltration or used without concentration. The column was equilibrated in “Equilibrium Buffer” (50 mM Tris-HC1, 150 mM NaCl, pH 7.0) before pumping the media over the column at a flow rate of 1-1.5 mL/minute. Following loading, the column was washed with 5 to 10 column volumes (CV) of Equilibration Buffer at a flow rate of 4 mL/minute. The column was then re-equilibrated with “Acetate Wash Buffer” (50 mM Sodium Acetate, 150 mM NaCl, pH 5.4).

Elution of bound protein from the column was performed at a flow rate of 1 mL/minute using three step elutions: (1) 50 mM Sodium Acetate, 150 mM NaCl, pH 3.4; (2) 50 mM Sodium Acetate, 150 mM NaCl, pH 3.2; and (3) 100 mM Glycine-HC1, pH 3.0. Fractions (1 mL/fraction) were collected into tubes containing 1 ml of “Formulation Buffer” (50 mM Sodium Acetate, 50 mM NaCl, pH 5.4) to raise the pH. The column was regenerated using 100 mM Glycine, pH 2.8 and then washed with dH₂O and stored in 20% ethanol.

Fractions containing protein, as determined by monitored by absorbance at 280 nm, were pooled and buffer exchanged into Formulation Buffer by overnight dialysis at 4° C. or by a spin-desalting column Concentration of the final protein solution was achieved by ultrafiltration using a 10 kDa concentrator. Any precipitate that may have formed during concentration or dialysis was removed by centrifugation at 2000×g for 30 minutes. The final sample was sterile filtered using a 0.22 mm cartridge.

The purified protein was characterized by: SDS-PAGE, analytical size exclusion chromatography (aSEC) and Western blot. Endotoxin levels were also measured. Purity was typically greater than 90% by aSEC and SDS-PAGE. The SDS-PAGE was shown in FIG. 6.

### Example 5

### In Vitro TFPI-Binding ELISA of GA200 and 56E4-GA200

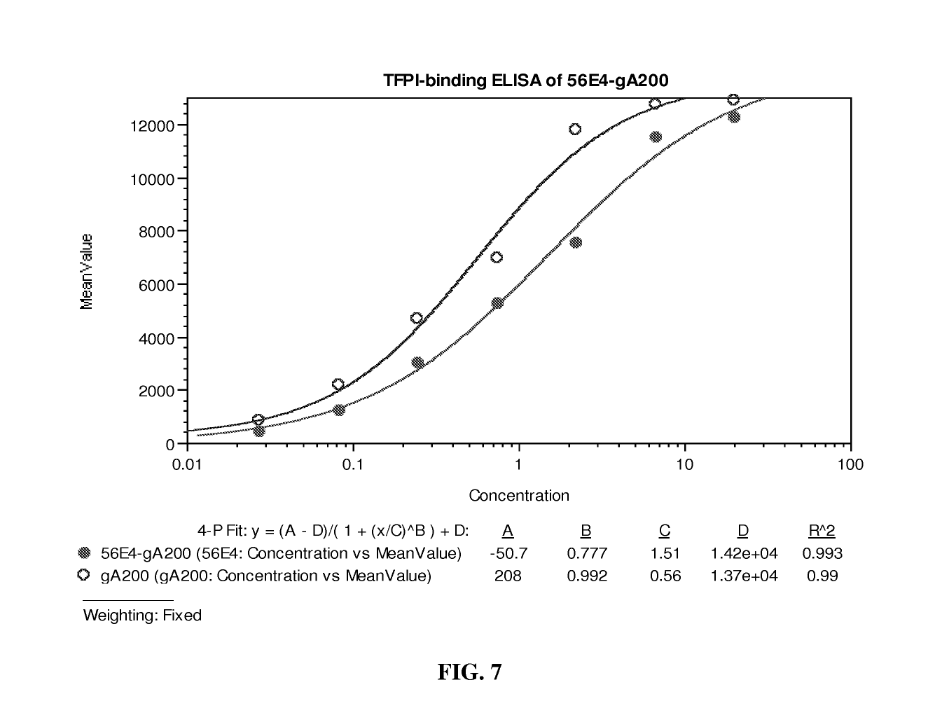

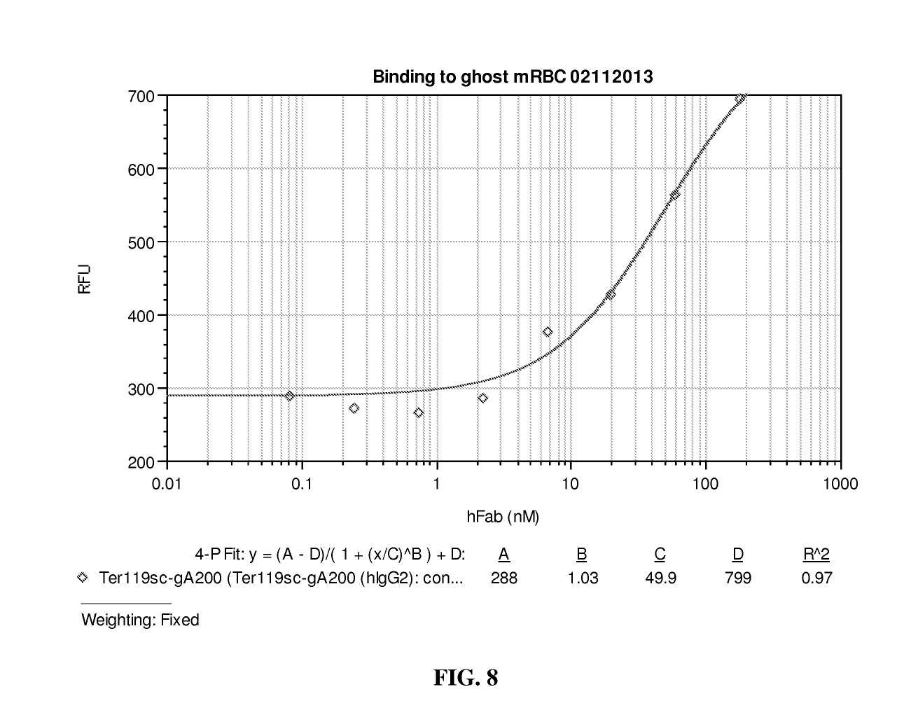

A Maxisorb 96-well plate (Nunc) was coated with 1 μg/mL of TFPI in PBS o/n at 4° C. The plate was blocked for 1 hour at room temperature in 5% non-fat dry milk PBS/0.5% Tween-20. Serial three-fold dilutions of undigested and digested antibodies were added to the wells (100 μL/well) and incubated for 1 hour at room temperature. The plates were washed 5 times in PBS-T. A secondary anti-Fab-HRP conjugated antibody was added (100 μL of a 1:10,000 dilution) for detection with an Amplex Red (Invitrogen) solution. The HSA-binding pro-drug antibody has slightly decreased binding on TFPI than its parental anti-TFPI antibody gA200 as can be seen in FIG. 7.

### Example 6

### RBC-Binding ELISA of TER119SA-GA200 Pro-Drug Antibody

ELISA was used to test pro-drug antibody binding on RBCs. The wells of a clear 96 well Maxisorp microtiter plate was coated with 100 μL, of mouse ghost RBCs resuspended in DPBS (without Ca or Mg) at a concentration of 10⁷/ml. Plates were sealed with tape and incubated overnight at 4° C. The wells were washed once with DPBST (DPBS+0.05% Tween 20) and then blocked with 5% Milk/DPBST for 1 hour at room temperature. Block buffer was discarded and 50 μL of diluted sample was added per well. Samples were serially diluted 1:3 in PBS. The plate was incubated for 1 hour at room temperature and then washed 5× quickly with DPBST. 100 μL of secondary antibody diluted 1:5000 in PBST (HRP-goat anti hFAB, Jackson ImmunoResearch, cat#109-035-097) was added per well. Plates were incubated for 1 hour at room temperature and then washed 5× with DPBST. HRP substrate (Amplex Red, Invitrogen A22177) was added and fluorescence readings at excitation wavelength of 485 nM and an emission wavelength of 595 nM were taken on a SpectraMax M2e (Molecular Devices). The pro-drug antibody bound to the RBCs at concentrations above 10 nM as can be seen in FIG. 8.

### Example 7

### Biacore™ Analysis of Anti-TFPI Pro-Drug Antibodies

Methods.

Human TFPI was immobilized on CM4 or CM5 chip using amine coupling kit based on manufacturer's instruction. Anti-TFPI pro-drug antibodies or parental anti-TFPI antibody were flowed through the system with 10 μg/mL antibody with or without 15 μg/mL human serum albumin (HSA). The binding level was measure at 2 seconds after completion of each injection. In kinetics assay, antibodies with a series of concentrations were injected, followed by 30-minute dissociation time. The dissociation and association rate of the antibodies were modeled using BiaEvaluation software.

Results.

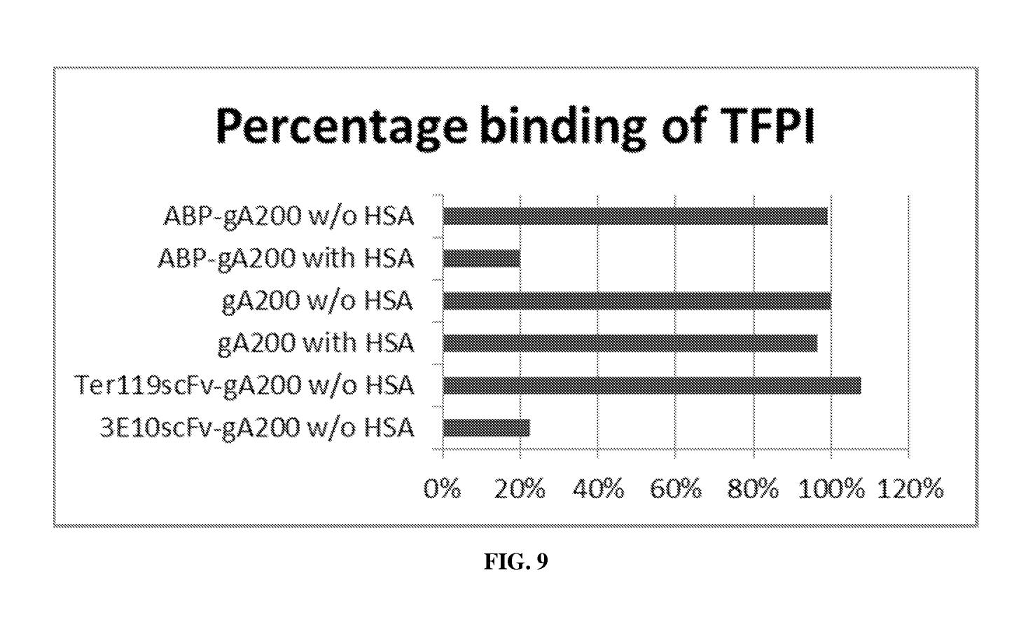

FIG. 9 shows different pro-drug antibodies binding to human TFPI. As shown in the FIG. 9, in the absence of HSA, ABP-gA200 pro-drug antibody binds to TFPI with 179 reflective unit (RU), while in the presence of HSA, the signal decreased 80% to 36 RU. Conversely, human albumin did not significantly affect the parental antibody gA200 binding on TFPI.

The RBC-binding pro-drug antibody, Ter119scFv-gA200, bound to TFPI at same level as gA200 while TF-binding pro-drug antibody, 3E10scFv-gA200, bound to TFPI with 22% residual level.

To further measure the binding of those pro-drug antibodies, a kinetics assay was conducted to measure affinity. As shown in Tables 1a-d, anti-TF pro-drug antibody, 3E10scFv-gA200, and anti-RBC pro-drug antibody, Ter119scFv-gA200, decreased binding on TFPI with 29.71-fold and 14.66-fold, respectively. The albumin binding pro-drug antibody, ABP-gA200, did not reduce binding to TFPI, but after mixed and incubated with HSA, its binding on TFPI also decrease 15.34-fold.

TABLE 1a
Results of Kinetic Assay on Anti-TFPI Pro-drug Antibodies Compared to
Unmodified Anti-TFPI Antibodies

| | | | | Affinity |
|---|---|---|---|---|
| | | | | decrease |
| Sample | kₐ | k_{d} | KD | (fold) |
| gA200 | 2.69E+06 | 4.76E−05 | 1.75E−11 | 1.00 |
| gA200 with HSA | 2.57E+06 | 4.65E−05 | 1.81E−11 | 1.04 |
| 3E10scFv-gA200 | 1.43E+05 | 7.40E−05 | 5.19E−10 | 29.71 |
| Ter119scFv-gA200 | 4.80E+05 | 1.23E−04 | 2.56E−10 | 14.66 |
| ABP-gA200 | 2.84E+06 | 6.03E−05 | 2.12E−11 | 1.21 |
| ABP-gA200 with HSA | 1.12E+05 | 3.00E−05 | 2.68E−10 | 15.34 |

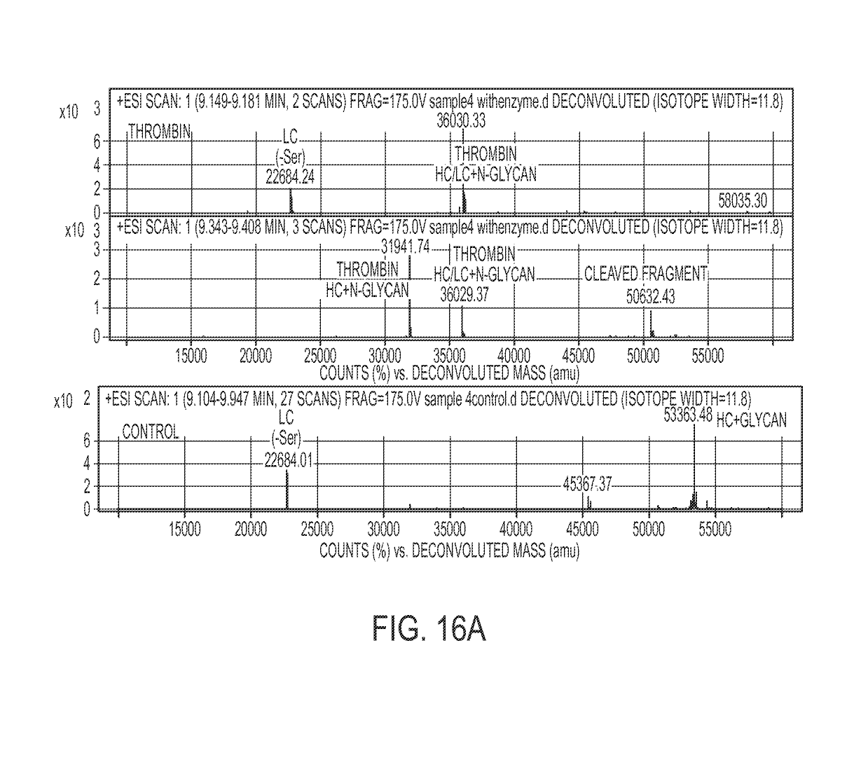

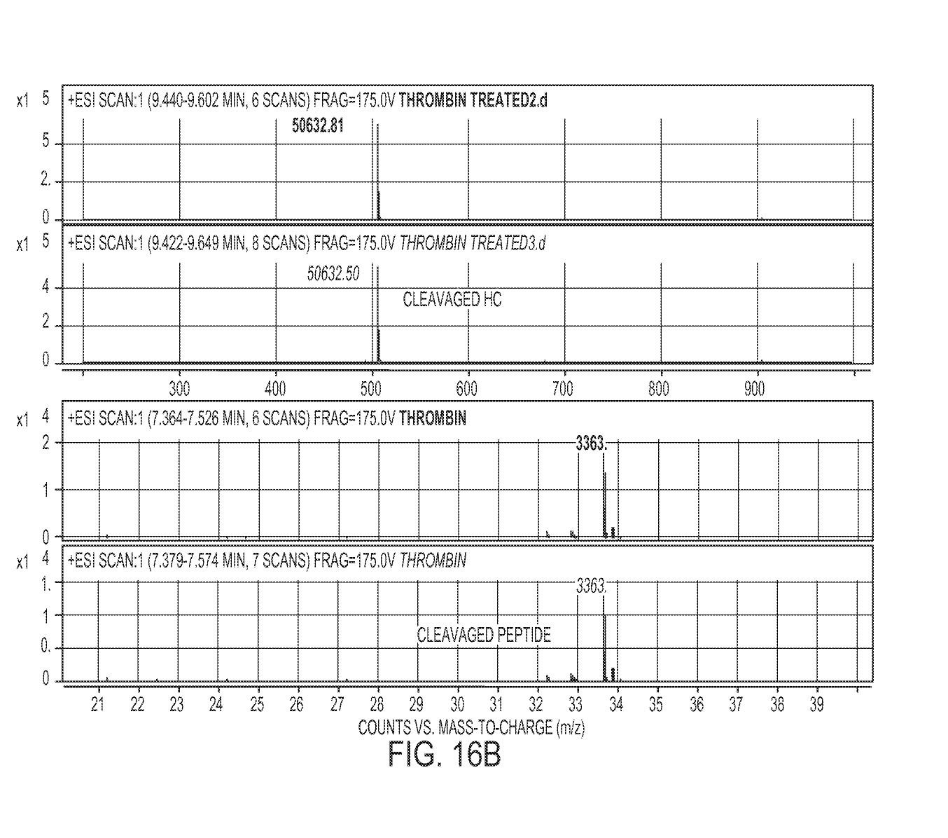

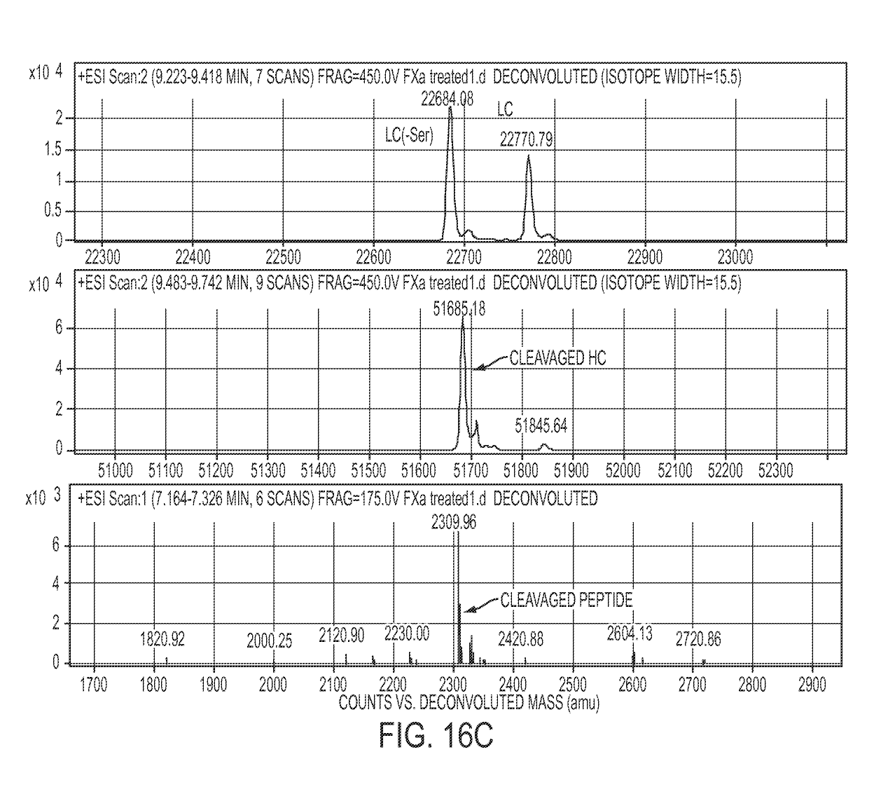

To further optimize the cleavage of ABP-gA200, the inventors had modified the cleavage site of ABP-gA200. Different cleavage sites were inserted between the albumin-binding peptide and the variable region of heavy chain. As shown in FIGS. 14A-B, spacers GGGGS were inserted around the cleavage sites. Whereas TPP-2651, TPP-2652, TPP-2653 and TPP2655 contained thrombin cleavage site, TPP-2654 contained a linker that can be cleaved by both FXa and thrombin. As shown in Table 1b, in the presence of human or monkey albumin, these antibodies decreased their binding to TFPI up to 28.6 fold for TPP- 2654 and 39.6 fold for TPP-2652 whereas the parental antibody gA200 had similar TFPI- binding affinity in the absence or presence of albumin TPP-2653 did not express with integrity, so its data was not shown. These well-expressed prodrug antibodies were purified and digested by proteases, either thrombin or Factor Xa. After removal of these proteases, the antibodies were analyzed using LC-MS. The results indicate the proteases cleaved the albumin-binding peptides. Representative data of TPP-2652 and TPP-2654 were shown in FIG. 16.

TABLE 1b
Results of Anti-TFPI Pro-drug Abs Compared
to Unmodified Anti-TFPI Abs

| | Affinity decrease (fold) | | |
|---|---|---|---|
| Affinity (KD) | | w/ | w/ |

| Antibody | alone | w/HSA | w/mkSA | alone | HSA | mkSA |
|---|---|---|---|---|---|---|
| TPP-2652 | 6.67E−12 | 1.20E−11 | 2.64E−10 | 1.0 | 1.8 | 39.6 |
| TPP-2654 | 1.75E−12 | 4.99E−11 | 2.63E−11 | 1.0 | 28.6 | 15.1 |
| TPP-2655 | 2.84E−12 | 1.36E−11 | 4.06E−11 | 1.0 | 4.8 | 14.3 |
| gA200 | 2.69E−12 | 2.01E−12 | 1.36E−12 | 1.0 | 0.7 | 0.5 |

To further balance the cleavage efficiency and masking function the anti-TFPI prodrugs, the inventors altered the linker length and truncated FR1 domain of antibody gA200. The amino terminal sequences of prodrug with ABP were shown in the FIGURES.

TABLE 1c
Results of crude anti-TFPI Pro-drug Abs
Compared to Unmodified Anti-TFPI Abs

| | IgG Conc. | TFPI | TFPI w/ 1 mg/ml | % masking by 1 mg |
|---|---|---|---|---|
| TPP # | (μg/ml) | w/o HSA | HSA | HSA |
| 2654 | 9.7 | 386 | 153 | 60.2% |
| 2981 | 1.5 | 123 | 27 | 77.9% |
| 2982 | 0.8 | 86 | 22 | 74.8% |
| 2983 | 1.1 | 118 | 46 | 61.1% |
| 2984 | 2.5 | 178 | 44 | 75.6% |
| 2985 | 2.4 | 188 | 47 | 75.3% |
| 2986 | 1.2 | 148 | 52 | 64.7% |
| 2987 | 2.1 | 185 | 36 | 80.8% |
| 2988 | 1.8 | 200 | 33 | 83.4% |
| 2989 | 2.5 | 183 | 87 | 52.4% |
| 2990 | 2.6 | 170 | 48 | 71.8% |
| 2991 | 1.9 | 144 | 34 | 76.1% |
| 2992 | 2.0 | 182 | 51 | 71.7% |
| 2993 | 2.5 | 178 | 29 | 83.5% |
| 2994 | 2.1 | 168 | 26 | 84.4% |
| 2995 | 0.9 | 94 | 48 | 48.4% |
| 2996 | 2.6 | 164 | 34 | 79.5% |
| 2997 | 2.1 | 157 | 31 | 80.5% |
| 2998 | 2.1 | 154 | 37 | 75.8% |
| 2999 | 3.3 | 184 | 29 | 84.3% |
| 3000 | 2.7 | 173 | 25 | 85.6% |
| 3001 | 1.2 | 72 | 29 | 59.2% |
| 3002 | nd | nd | nd | na |
| 3003 | 3.3 | 261 | 84 | 67.6% |
| 3004 | 2.2 | 193 | 35 | 81.9% |
| 3005 | 1.5 | 159 | 25 | 84.2% |
| 2652 | 4.6 | 310.6 | 95 | 69.4% |
| gA200 | 1.0 | 110.5 | 124.9 | −13.0% |

TABLE 1D
Results of purified Anti-TFPI Pro-drug Abs Compared to Unmodified Anti-TFPI Abs

| RU | | | | % masking | | | | |
|---|---|---|---|---|---|---|---|---|
| | Alone | w/ MkSA | w/ HSA | | Alone | w/ MkSA | w/ HSA | Sup w/HSA |
| HBS | 0 | 2.6 | 2.9 | HBS | 0% | 0% | 0% | 0 |
| gA200 | 124.9 | 121.1 | 120.6 | gA200 | 0% | 3% | 3% | 0 |
| 2987 | 63.8 | 19.6 | 17.2 | 2987 | 0% | 69% | 73% | 80.8% |
| 2988 | 46 | 10 | 6.6 | 2988 | 0% | 78% | 86% | 83.4% |
| 2993 | 34.6 | 5.4 | 6 | 2993 | 0% | 84% | 83% | 83.5% |
| 2994 | 33.9 | 5.9 | 6.7 | 2994 | 0% | 83% | 80% | 84.4% |
| 2998 | 51.7 | 22.6 | 24.2 | 2998 | 0% | 56% | 53% | 75.8% |
| 2999 | 67.7 | 35.1 | 38.1 | 2999 | 0% | 48% | 44% | 84.3% |
| 3000 | 27.5 | 5.5 | 5.1 | 3000 | 0% | 80% | 81% | 85.6% |
| 3002 | 14.2 | 5.3 | 5 | 3002 | 0% | 63% | 65% | nd |
| 3004 | 59.5 | 20.6 | 20.8 | 3004 | 0% | 65% | 65% | 81.9% |
| 3005 | 50.2 | 7.6 | 6.9 | 3005 | 0% | 85% | 86% | 84.2% |
| 2652 | 38.5 | 9.9 | 9.8 | 2652 | 0% | 74% | 75% | 69.4% |
| 2654 | 20.2 | 4.8 | 4 | 2654 | 0% | 76% | 80% | 60.2% |

### Example 8

### Thrombin Generation Assay for Anti-TFPI Pro-Drug Antibodies

Thrombin generation assay (TGA) of TFPI pro-drug antibodies was conducted using human HemA Plasma. Platelet poor plasma (PPP) reagent and calibrator were reconstituted with 1 mL of distilled water. A 1:2 serial dilution of anti-TFPI pro-drug antibodies, starting from 100 nM of final concentration to 1.56 nM, was added in HemA human plasma. The plasma only sample was used as control. In a 96-well TGA plate, 20 μL of PPP reagent or calibrator was added to each well followed by adding 80 μL of plasma sample containing different concentration of anti-TFPI pro-drug antibodies. The plate was placed in a TGA instrument, and then the instrument automatically dispensed 20 μL of FluCa (Fluo substrate+CaC12) in each well. The thrombin generation was measured for 60 minutes. When Ter119scFv-gA200 was tested in TGA, HemA Plasma was spiked in mouse RBC ghost. Ter119scFv-gA200 was incubated with mouse RBC-GOLD at room temperature for 15 min

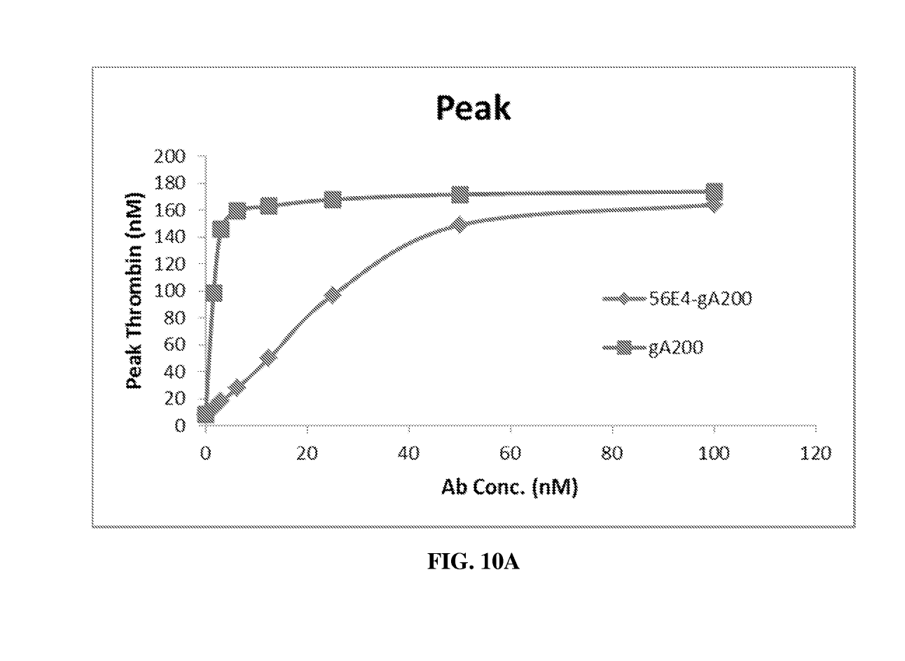

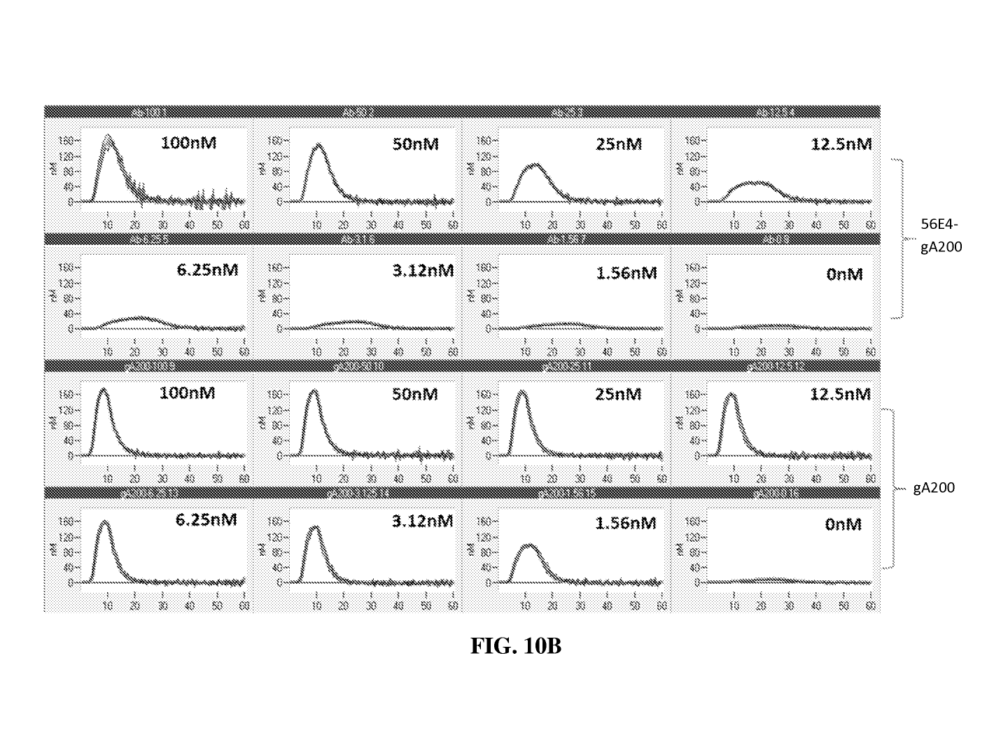

As shown in FIGS. 10A-B, 56E4-gA200 generated lower thrombin peak than its parental antibody gA200, indicating the human albumin in the plasma might reduce the activity of anti-TFPI. The shape of thrombin generated by 56E4gA200 was a low and broad peak also indicates that the antibody was less active in the time zero and might become activated by generated FXa or thrombin.

Feasibility of converting the prodrug TFPI antibody to more active TFPI antibody under physiologic conditions was modeled by 1) the ability of exogenous thrombin (at physiologic levels) to increase TFPI Ab-mediated TGA responses, and 2) by generating FXa and thrombin in the TGA reaction and monitoring the subsequent increase in TFPI antibody-induced response.

Exogenous thrombin addition would directly assess the susceptibility of prodrug TFPI antibody to the enzyme at levels potentially achievable physiologically, and was performed by pre-incubating 1200 nM Ab with 0.5 to 2.5 U/mL thrombin for 1 hr., followed by inactivation of the thrombin with the thrombin-specific irreversible inhibitor hirudin at 0.5 to 2.5 U/mL for 1 hr. To gauge the effect of hirudin carryover in the TGA reaction, buffer replaced thrombin to assess the effect of hirudin carryover into the TGA reactions. In some cases, irrelevant Ab or the parental antibody gA200 (without albumin masking sequences or protease-susceptible sites) were used in place of pro-TFPI Ab as an control. The antibody-thrombin-hirudin mixtures were serially diluted to 10 to 100 nM Ab concentrations, and the mixtures were additionally diluted 1:10 in the TGA reaction. TGA reactions were performed as described above except that the initiator used was PPP-Low, containing 1 pM TF-4 nM platelets. The TGA results with irrelevant antibody were subtracted from those with prodrug TFPI antibody.

TGA profiles of pro-TFPI Ab TPP-2654 before and after protease-cleavage are shown in FIG. 10C. The TGA response of parental gA200 was unaltered by thrombin incubation, while TPP-2654, which contained both a thrombin- and a FXa-susceptible cleavage site showed increased response when preincubated with thrombin. However, the peak TGA response was less than with gA200, suggesting either that complete unmasking of TFPI Ab activity may require the presence of higher thrombin levels or an optimized protease-susceptible sites to increase efficiency of protease cleavage.

Titration experiments with a range of thrombin concentrations (0.5 to 2.5 U/mL) established that maximum conversion of prodrug TFPI antibody to more active TFPI antibody can be achieved at 1 U/mL thrombin, levels potentially achievable in vivo (FIG. 10D).

To assess whether FXa or thrombin can more efficiently convert prodrug antibody to active TFPI antibody, particularly in prodrug antibody containing both protease-susceptible sites, effect of increasing FXa and thrombin in situ was assessed. FXa generation in situ was increased by increasing the concentration of TF used as initiator. In these experiments TF concentration was varied from 1 pM to 5 pM by using either PPP-Low (1 pM TF-4 μM PL) or PPP Reagent (5 pM TF-4 μM PL) as initiators in the TGA reactions. Increasing TF would increase FXa through the direct action of TF-FVIIa, and increased FXa would, in turn, increase thrombin generation. TGA reactions were performed as described above, and the results were analyzed by comparing the difference in response between TGA reactions with 1 pM vs 5 pM TF.

FXa- and thrombin-susceptibility of TPP-2654 was evident in the increased difference in peak thrombin response (delta peak) between 1 pM and 5 pM TF intiator (FIG. 10E), particularly with prodrug TFPI antibody pre-incubated with 2.5 U/mL thrombin. The ability of increasing peak thrombin responses of pro-TFPI Ab TPP-2654 further beyond the response attained with initial thrombin pre-incubation indicated that FXa cleavage may further contribute to the complete unmasking of albumin binding peptides.

The TGA responses of a pro-TFPI Ab (TPP-2652) where the masking albumin binding peptides are removed by thrombin cleavage are shown in FIGS. 10E-G. Thrombin susceptibility of TPP-2652 is shown in FIG. 10F, indicating that at the maximal concentration of thrombin tested (2.5 U/mL), only a small increase in TGA response was detected at the highest level of antibody tested. Increasing FXa (and thrombin) by increasing the TF concentration used to initiate the TGA only slightly increased the TGA response of TPP-2652 further (FIG. 10F). This is in contrast to the large increase in thrombin response with TPP-2654 when thrombin-pretreated TPP-2654 was further exposed to FXa generated with 5 pM TF (compare FIG. 10E with FIG. 10G).

These results suggest that the different protease-susceptible sites can affect the conversion of prodrug-TFPI antibody to active TFPI Ab.

### Example 9

### Fxa Assay for Anti-TFPI Pro-Drug Antibodies

### Materials and Methods

The following reagents were used for FXa assay:

Assay Buffer: 1× buffer is 25 mM Hepes 7.4, 100 mM NaCl, 5 mM CaCl₂, 0.1% BSA.

TFPI—R&D (Cat#2974-PI, MW ˜35 kDa). TFPI was reconstituted to 100 μg/mL (2.86 μM) by adding 10 μL of 25 mM Tris and 150 mM NaCl, pH 7.5 following the product insert instructions. The 2.86 μM stock was diluted 1/143 to generate a 20 nM working stock.

FXa—Haematologic Technologies (Cat# HCX-0060, MW—58.9 kDa) Stock 2 μM aliquots were previously made in assay buffer and stored at −80° C. The 2 μM stock was diluted 1/1000 for a 2 nM working stock.

S-2765—Chromogenix (Cat # S-2765, MW—714.6 Da) A 5 mM working stock was generated by dissolving the 25 mg of lyophlized material in 7 mL distilled water. The 5 mM working stock was added directly into the assay wells.

A 4× dose curve of anti-TFPI antibodies is generated in assay buffer. 60 μL of each antibody concentration was combined with a 4× (20 nM) concentration of TFPI. The antibody/TFPI mixture is incubated for 30 minutes at room temperature. 120 μL of a 2× (2 nM) concentration of FXa is added to the Ab/TFPI mixture and incubated for 30 minutes at room temperature. The Ab/TFPI/FXa mixture is then transferred to an assay plate in duplicate at 100 μL per well followed by 20 μL of 5 mM substrate. The plate is immediately read kinetically at 405 nm for 3 minutes in a Molecular Devices SpectraMax plate reader. When albumin binding anti-TFPI pro-drug antibody, 56E4-gA200, was tested, 34 μL 8× anti-TFPI antibodies was combined with 34 μL albumin of different concentration in 96-well round bottom polypropylene plate. The solution was then incubated for 15 minutes at room temperature and 68 μL of 20 nM (4×) TFPI was added to the α-TFPI Ab/albumin.

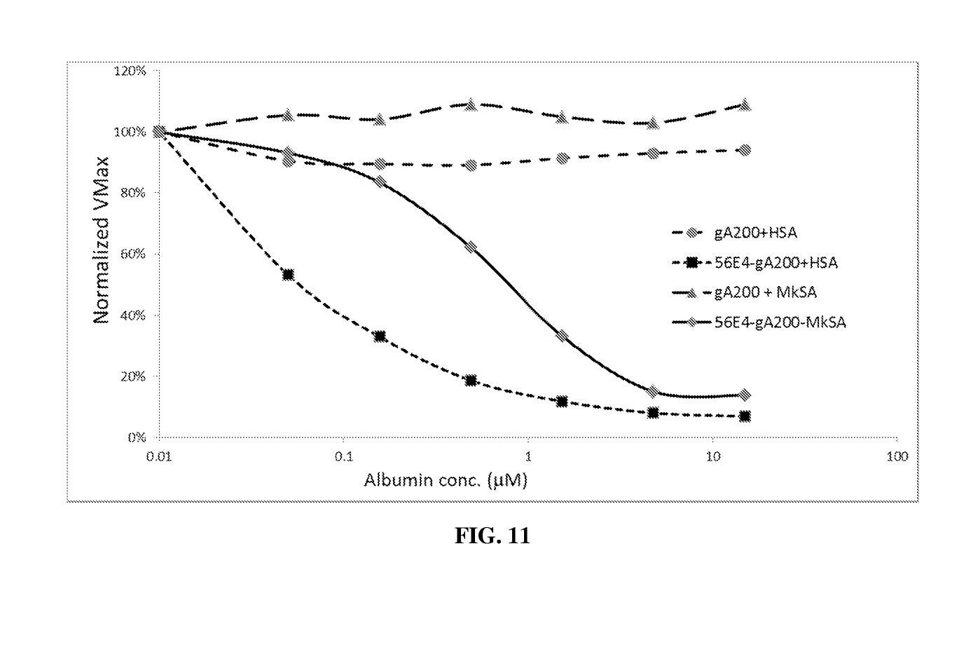

As shown in FIG. 11, Vₘₐₓ of gA200 was not affected by either human or monkey albumin. The albumin-binding pro-drug antibody, 56E4-gA200, decreased Vₘₐₓ with increased concentration of albumin. When the concentration of human or monkey albumin reached 5 μM, the anti-TFPI activity of the pro-drug antibody was 90% inhibited.

### Example 10

### Protease Digestion of Anti-TFPI Pro-Drug Antibodies

The Novagen Thrombin cleavage capture kit (69022-3) and the Novagen Factor Xa kit (69037-3) were used for protease cleavage and protease depletion of the prodrugs.

Biotinylated thrombin was used for prodrug cleavage as briefly described below.

The 50 μL reactions for each prodrug contained 5 μg of prodrug, 5 μL 10× kit thrombin cleavage/capture buffer, 1 unit thrombin, deionized water to 50 μL. The reactions were incubated at 37° C. for 1 hr. After the cleavage reaction, the biotinylated thrombin was removed with streptavidin agarose (supplied in the kit) using a ratio of 16 ml settled resin (32 ml of the 50% slurry) per unit of enzyme. After the agarose was added to the reaction tube, the tube was incubated at room temperature for 30 min with gentle shaking. The entire reaction was transferred to the kit supplied sample cups with spin filters. Then the tubes were centrifuged at 500×g for 5 minutes. The filtrate in the collection tube contains the cleaved prodrug, free of biotinylated thrombin.

When Factor Xa was used for prodrug cleavage, 50 μL reactions for each prodrug contained 5 μg of prodrug, 5 μL 10× kit Factor Xa Cleavage/Capture Buffer, 1 unit Factor Xa, and deionized water to 50 μL total volume. The reactions were incubated at 37° C. for 1 hr. After the cleavage reaction, Factor Xa was removed with Xarrest™ Agarose (supplied in the kit). Xarrest Agarose was first equilibrated by adding 11 volume of 1× Factor Xa Cleavage/Capture Buffer per settled resin volume of Xarrest Agarose. The Xarrest agarose was centrifuged at 1000×g for 5 min. The supernatant was removed and discarded. The agarose was resuspended in 10 volume of 1× Factor Xa Cleavage/Capture Buffer, and centrifuged at 1000×g for 5 mM Supernatant was removed and discarded. One settled resin vol 1× Factor Xa Cleavage/Capture Buffer was added to the tube and the resin was fully resuspended. The prepared Xarrest Agarose was transferred to a sample cup of a 2 ml Spin Filter (included with kit). The entire volume of prodrug cleavage reaction was added to the prepared Xarrest Agarose. The tube was incubated at room temperature for 5 min and centrifuged at 1000×g for 5 mM to remove the Xarrest Agarose. Bound Factor Xa is retained in sample cup, and the cleaved prodrug flows into the filtrate tube during centrifugation.

### Example 11

### LC-Ms of Anti-TFPI Pro-Drug Antibodies

LC-MS analysis for antiTFPI pro-drug antibodies TPP-2652 and TPP-2654 were conducted under either intact or reduced condition. For intact protein, directly load 2 μg to the PLRP column. For reducing sample, prior load to PLRP column, the testing samples have been treated with 10 mM DTT, 37° C. for 30 minutes.

The LC separation was carried using Agilent 1200 Capillary LC System with PLRP-S (8 μm 4000A, 0.3×150 mm) at 70° C. The buffer systems for LC were: A: Water with 0.1% Formic Acid+0.01% TFA, B: Acetonitrile with 0.1% Formic Acid+0.01% TFA, flow rate 10 μL/min. The gradient: 10% B in 2 mM, to 90% B in 15 min, 90% B for 5 mM, 10% B equilibration for 10 min

The MS analysis was performed using Agilent 6520 Q-TOF system. The conditions were DualEsi source, gas temp: 350° C., drying gas: 7 psi, nebulizer: 10 psi, scan range: 500-3000 amu, 1 spectra/s. Two experiments per cycle: 3500v, 175v fragmentor, 65v skimmer for reduced forms and 4000v, 350v fragmentor, 100v skimmer for intact protein. Reference ions: 1221.990637 and 2421.91399 amu, 50 ppm window, MM 1000 The prodrug antibodies were purified and digested by proteases, either thrombin or Factor Xa. After removal of these proteases, the antibodies were analyzed using LC-MS. The results indicate the proteases cleaved the albumin-binding peptides. Representative data of TPP-2652 and TPP-2654 were shown in FIGS. 16A-C.

TABLE 2
Key for FIG. 13

| HEAVY CHAIN | LIGHT CHAIN |
|---|---|
| A1 | A3 |
| A2 | A3 |
| A4 | A3 |
| B1 | B2 |
| B3A | A3 |
| B3B | A3 |
| B4 | A3 |
| B5 | A3 |
| B6 | A3 |
| B7 | B7A |
| B8 | B8A |
| B9 | B9A |
| B10 | B2 |
| B11 | A3 |
| B12 | A3 |
| B13 | A3 |
| B14 | A3 |
| B15 | A3 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An antibody comprising:
(a) a first variable domain comprising a first light and a first heavy chain variable region, said first variable domain binding to Tissue Factor Pathway Inhibitor (TFPI);
(b) a masking domain linked to the amino terminus of said first light and/or first heavy chain variable region; and
(c) a protease cleavable linker interposed between said first light and/or first heavy chain variable region and said masking domain.

2. The antibody of claim 1, wherein said protease cleavable domain is a thrombin, plasmin, Factor VIIa or Factor Xa cleavage site.

3. The antibody of claim 1, wherein said masking domain comprises a second variable domain comprising a second light and a second heavy chain variable region.

4. The antibody of claim 1, wherein said antibody is an IgG1, an IgG2, an IgG3, an IgG4, an IgM, an IgA 1, an IgA2, a secretory IgA, an IgD, and an IgE antibody.

5. The antibody of claim 1, wherein said antibody is a human or humanized antibody.

6. The antibody of claim 1, wherein said antibody is a single-chain antibody.

7. The antibody of claim 1, wherein said antibody is bivalent and comprises two masking domains, one linked to the amino terminus of each first light chain variable region.

8. The antibody of claim 1, wherein said antibody is bivalent and comprises two masking domains, one linked to the amino terminus of each first heavy chain variable region.

9. The antibody of claim 1, wherein said antibody is bivalent and comprises four masking domains, one linked to the amino terminus of each first light chain variable region and each first heavy chain variable region.

10. The antibody of claim 9, wherein two of the masking domains are a second light chain variable region, and two of the masking domains are a second heavy chain variable region, wherein said second light and heavy chain variable regions form a second variable domain.

11. The antibody of claim 3 or 10, wherein said second variable domain binds to tissue factor (TF), a red blood cell, or albumin.

12. The antibody of claim 1, 7 or 8, wherein said masking domain is albumin binding peptide or protein.

13. An expression vector comprising a coding region for an antibody according to claims 1-12 under the control of a promoter.

14. A cell comprising an expression vector according to claim 13.

15. A pharmaceutical formulation comprising an antibody according to claims 1-12 formulated with a pharmaceutically acceptable buffer, carrier or diluent.

16. A method of treating a coagulation disorder in a subject comprising administering to said subject an antibody comprising:
(a) a first variable domain comprising a first light and a first heavy chain variable region, said first variable domain binding immunologically to Tissue Factor Pathway Inhibitor (TFPI);
(b) a masking domain linked to the amino terminus of said first light and/or first heavy chain variable region; and
(c) a protease cleavable linker interposed between said first light and/or first heavy chain variable region and said masking domain,
in an amount effective to promote coagulation in said subject.

17. The method of claim 16, wherein said protease cleavable domain is a thrombin, plasmin, Factor VIIa or Factor Xa cleavage site.

18. The method of claim 16, wherein said masking domain comprises a second variable domain comprising a second light and a second heavy chain variable region.

19. The method of claim 16, wherein said subject is a human.

20. The method of claim 16, wherein said subject is a non-human mammal.

21. The method of claim 16, wherein said antibody is a single-chain antibody.

22. The method of claim 16, wherein said antibody is bivalent and comprises two masking domains, one linked to the amino terminus of each first light chain variable region.

23. The method of claim 16, wherein said antibody is bivalent and comprises two masking domains, one linked to the amino terminus of each first heavy chain variable region.

24. The method of claim 16, wherein said antibody is bivalent and comprises four masking domains, one linked to the amino terminus of each first light chain variable region and each first heavy chain variable region.

25. The method of claim 24, wherein two of the masking domains are a second light chain variable region, and two of the masking domains are a second heavy chain variable region, wherein said second light and heavy chain variable regions form a second variable domain.

26. The method of claim 18 or 25, wherein said second variable domain binds to tissue factor (TF), a red blood cell or albumin.

27. The method of claim 16, 22 or 23, wherein said masking domain is albumin binding protein.

28. The method of claim 16, wherein said subject suffers from trauma, hemophilia or cancer.

28. The method of claim 16, wherein said subject suffers from hemophilia A or B.

30. The method of claim 16, wherein said antibody is administered systemically.

31. The method of claim 16, wherein said antibody is administered locally or regionally to a site of bleeding.

32. The method of claim 16, wherein said antibody is administered subcutaneously, intravenously or intra-arterially.

33. The antibody of claim 16, wherein said antibody is an IgG1, an IgG2, an IgG3, an IgG4, an IgM, an IgA 1, an IgA2, a secretory IgA, an IgD, and an IgE antibody.

34. The method of claim 16 wherein said antibody is a human or humanized antibody.

35. The method of claim 16, wherein said antibody binds to Kunitz domain 2 of human tissue factor pathway inhibitor.
